(19) United States
(12) Patent Application Publication
(10) Pub. No.: US 2023/0210797 A1
(43) Pub. Date: Jul. 6, 2023

(54) PREVENTING AND TREATING HYPOGLYCEMIA

(71) Applicant: DIAMYD MEDICAL AB, Stockholm (SE)

(72) Inventors: Daniel ESPES, Uppsala (SE); Per-Ola CARLSSON, Storvreta (SE)

(21) Appl. No.: 17/630,137

(22) PCT Filed: Aug. 7, 2020
(86) PCT No.: PCT/SE2020/050771

§ 371 Date: Jan. 25, 2022

(30) Foreign Application Priority Data
Aug. 9, 2019 (SE) 1950921-5
May 4, 2020 (SE) 2050506-1

(51) Int. Cl. A61K 31/197; A61K 45/06; A61K 38/28; A61K 31/64; A61P 3/08

(52) U.S. Cl. CPC A61K 31/197; A61K 45/06; A61K 38/28; A61K 31/64; A61P 3/08

(57) ABSTRACT

The present invention relates to Gamma-amino butyric acid, optionally in combination with a Positive Allosteric Modulator of a GABA-receptor, for use in a method for preventing, or reducing risk of, hypoglycemia in a subject.

### FIELD OF THE INVENTION

The present invention relates to methods of preventing hypoglycemia, reducing risk of hypoglycemia, and treating impaired counter regulatory response to hypoglycemia, as well as compounds useful in such treatments.

### BACKGROUND

In a healthy individual, hypoglycemia is effectively countered by a sequence of hormones involving suppression of insulin release, activation of rapid acting glucagon and epinephrine, followed by release of slow-acting growth hormone and cortisol. This hormonal response acts to increase glucose production and to decrease peripheral glucose utilization, leading to increased glucose delivery to the brain.

In type 1 diabetes and to some extent in type 2 diabetes, the counterregulatory response is impaired due to three main factors 1) an inability to clear insulin from the system, 2) reduced or absent glucagon release and 3) reduced autonomic response to hypoglycemia. It has further been shown that patients that are undergoing intense insulin treatment have an increased impairment of the counterregulatory response (McCrimmon & Sherwin, 2010). Recurring hypoglycaemic events have been shown to lead to impaired hypoglycemia awareness in both patients with type 1 diabetes and in healthy individuals (Davis et al., 1992) (Heller & Cryer, 1991) characterized by a higher threshold (lower glucose) for release of counterregulatory response and initiation of symptoms.

Besides insulin, certain anti-diabetic medications such as sulfonylureas have been associated with a greater risk for hypoglycemia. Also bariatric surgery as well as drugs such as beta-blockers and Selective Serotonin Reuptake Inhibitors (SSRIs) seem to increase the risk of hypoglycemia and hypoglycaemic unawareness (White, 2007). Other factors associated with counterregulatory response are gender, age and physical activity. Older age is associated with a less intense counterregulatory response whereas female gender has been associated with a reduced regulatory response but less blunting due to antecedent hypoglycemia. Exercise has also been associated with a blunted counterregulatory response.

Animal studies have linked an attenuated counterregulatory response to increased GABA signalling in key brain glucose-sensing sites, such as the ventromedial hypothalamus (Chan et al., 2008). In addition, lower measured cortical GABA receptor binding and lower GABA receptor expression in the cerebral cortex has been observed in diabetic mice and mice experiencing hypoglycemia (Antony et al, 2010). In humans, a GABA antagonistic drug, modafinil, was tested in 14 healthy adults and was shown to reduce noradrenergic response following hypoglycemia while other hormones were unaffected. In another experiment, patients with type 1 diabetes treated with the positive GABA receptor modulator alprazolam experienced an increased attenuation of counter regulatory response following exercise. Also, in healthy individuals it was shown that treatment with alprazolam resulted in significant blunting of the counter regulatory response (Hedrington et al., 2010) as well as inhibited the neuroendocrine response to insulin-induced hypoglycemia (Giordano et al., 2003)). A study in healthy men showed that protracted use of orally ingested GABA significantly blunted the growth hormone response to insulin induced hypoglycemia. These studies suggest that increased GABAergic signalling could be associated with attenuated counter regulatory response and risk for hypoglycemia.

WO2009/114718 describes a method of treating hypoglycemia comprising administering an effective amount of a compound that activates an ionotropic glutamate receptor to stimulate glucagon release. The method is thus for treating an existing hypoglycemia by stimulating glucagon release to acutely increase circulating glucagon levels by administering GABA supposedly acting on glutamate receptors. WO2009/114718 suggests that GABA may be expected function as a compound that activates an ionotropic glutamate receptor to stimulate glucagon release but contains no experimental data or theoretic reasoning to substantiate this suggestion.

### SUMMARY OF THE INVENTION

Surprisingly, in the present invention we observe a normalization of the counter regulatory response in long-standing type 1 diabetes patients following treatment with oral GABA. In five patients with no measurable endogenous insulin production, GABA treatment downregulated the basal cortisol and norepinephrine levels and restored the counterregulatory response when hypoglycemia was induced. There is also an observed increase in glucagon and growth hormone response during hypoglycemia.

The present invention thus in a first aspect relates to Gamma-amino butyric acid (GABA) for use in a method for preventing, or reducing risk of, hypoglycemia in a subject.

In a further aspect, the invention relates to GABA in combination at least one compound selected from the group consisting of Positive Allosteric Modulators of a GABA-receptor (PAM), Selective Serotonin Reuptake Inhibitors (SSRIs) and dehydroepiandrosterone, for use in a method for preventing, or reducing risk of, hypoglycemia in a subject.

In one embodiment, said subject suffers from type 1 diabetes, type 2 diabetes, insulin-dependent diabetes mellitus, and/or impaired endogenous insulin production.

In one embodiment, said subject has a fasting plasma level of c-peptide of <0.01 nmol/L before treatment according to the invention. In one embodiment, said subject has a maximum (Cₘₐₓ) plasma level of c-peptide in a Mixed Meal Tolerance Test of <0.01 nmol/L before treatment according to the invention.

In one embodiment, GABA is for use in preventing, or reducing risk of, insulin-induced hypoglycemia in said subject.

In one embodiment, GABA is for use in preventing, or reducing risk of, non-insulin-induced hypoglycemia in said subject.

In one embodiment, said non-insulin-induced hypoglycemia is induced by sulfonylurea, beta blockers, Selective Serotonin Re-uptake Inhibitors (SSRI:s), exercise, islet transplants, and/or bariatric surgery.

In one embodiment, said non-insulin-induced hypoglycemia is induced by sulfonylurea.

In a further aspect, the invention relates to insulin for use in medicine in combination with GABA, and optionally at least one compound selected from the group consisting of Positive Allosteric Modulators of a GABA-receptor (PAM), Selective Serotonin Reuptake Inhibitors (SSRIs) and dehydroepiandrosterone, for use according to the invention.

In a further aspect, the invention relates to sulfonylurea for use in medicine in combination with GABA, and optionally at least one compound selected from the group consisting of Positive Allosteric Modulators of a GABA-receptor (PAM), Selective Serotonin Reuptake Inhibitors (SSRIs) and dehydroepiandrosterone, for use according to the invention.

In a further aspect, the invention relates to Gamma-amino butyric acid (GABA) for use in a method for treating defects in hypoglycemia counterregulation in a subject.

In a further aspect, the invention relates to Gamma-amino butyric acid (GABA) in combination with at least one compound selected from the group consisting of Positive Allosteric Modulators of a GABA-receptor (PAM), Selective Serotonin Reuptake Inhibitors (SSRIs) and dehydroepiandrosterone, for use in a method for treating defects in hypoglycemia counterregulation in a subject.

In a further aspect, the invention relates to Gamma-amino butyric acid (GABA) for use in a method for increasing the time spent in normal blood glucose range (4-10 mmol/L) or target blood glucose range (4-8 mmol/L) by a subject.

In one embodiment, said subject suffers type 1 diabetes, type 2 diabetes, insulin-dependent diabetes mellitus, and/or impaired endogenous insulin production.

In one embodiment, the subject has c-peptide levels that are undetectable in a standard assay, i.e. <0.01 nmol/L.

In a further aspect, the invention relates to a pharmaceutical composition comprising insulin and GABA, and optionally at least one compound selected from the group consisting of Positive Allosteric Modulators of a GABA-receptor (PAM), Selective Serotonin Reuptake Inhibitors (SSRIs) and dehydroepiandrosterone, for use according to the invention.

In a further aspect, the invention relates to pharmaceutical composition comprising sulfonylurea and GABA, and optionally at least one compound selected from the group consisting of Positive Allosteric Modulators of a GABA-receptor (PAM), Selective Serotonin Reuptake Inhibitors (SSRIs) and dehydroepiandrosterone, for use according to the invention.

In the above aspects, GABA may be administered in a dose of 200 mg to 1200 mg per day, such as 600 mg per day.

In one aspect, the use of GABA in preventing, or reducing risk of, hypoglycemia in a subject is part of a treatment for treatment or prevention of Hyperglycemia-Associated Autonomic Failure (HAAF).

The present invention also relates to analogous therapeutic and pharmaceutical use of a GABA receptor agonist, such as GABA_{A} or GABA_{B} receptor agonists as defined below. Thus, in further aspects, GABA is substituted with another GABA receptor agonist in the invention as described above.

In some embodiments GABA, or the GABA receptor agonist, is administered to the subject one hour or less before a meal to lower risk of hypoglycemia after a meal. In some embodiments GABA, or the GABA receptor agonist, is administered to the subject one hour or less before going to bed to lower risk of hypoglycemia during sleep.

In some embodiments of the invention as described herein, the PAM is a benzodiazepine, such as alprazolam or diazepam. The daily dose of the PAM is preferably less than 100% of the Defined Daily Dose (1 mg for alprazolam, 10 mg for diazepam), such as less than or about 50% of the Defined Daily Dose, such as about 10%-25% of the Defined Daily Dose.

### Definitions

The term “GABA receptor agonist” refers generally, as used herein, to a compound that directly enhances the activity of a GABA_{A} or GABA_{B} receptor relative to the activity of the GABA receptor in the absence of the compound. “GABA receptor agonists” useful in the invention described herein include compounds such as GABA, muscimol, thiomuscimol, cis-aminocrotonic acid (CACA), homotaurine, bamaluzole, gabamide, GABOB, gaboxadol, ibotenic acid, isoguvacine, isonipecotic acid, phenibut, picamilon, progabide, quisqualamine, progabide acid (SL 75102).

The term “PAM” or “Positive Allosteric Modulator” refers to Positive allosteric modulators (PAMs) of GABA_{A} and/or GABA_{B} receptors and are well known to those of skill in the art. Illustrative PAMS include, but are not limited to alcohols {e.g., ethanol, isopropanol), avermectins {e.g., ivermectin), barbiturates {e.g., phenobarbital), benzodiazepines, bromides {e.g., potassium bromide, carbamates {e.g., meprobamate, carisoprodol), chloralose, chlormezanone, clomethiazole, dihydroergolines {e.g., ergoloid (dihydroergotoxine)), etazepine, etifoxine, imidazoles {e.g., etomidate), kavalactones (found in kava), loreclezole, neuroactive steroids {e.g., allopregnanolone, ganaxolone), nonbenzodiazepines (e.g., zaleplon, Zolpidem, zopiclone, eszopiclone), petri chloral, phenols (e.g., propofol), piped dinediones (e.g., glutethimide, methyprylon), propanidid, pyrazolopyridines (e.g., etazolate), quinazolinones (e.g., methaqualone), skullcap constituents (e.g. constituents of Scutellaria sp. including, but not limited to flavonoids such as baicalein), stiripentol, sulfonylalkanes (e.g., sulfonmethane, tetronal, trional), valerian constituents (e.g., valeric acid, valerenic acid), and certain volatiles/gases (e.g., chloral hydrate, chloroform, diethyl ether, sevoflurane). The PAMs used in combination with the GABA receptor activating ligands may exclude alcohols, and/or kavalactones, and/or skullcap or skullcap constituents, and/or valerian or valerian constituents, and/or volatile gases. The PAM may comprise an agent selected from the group consisting of a barbituate, a benzodiazepine, a quinazolinone, and a neurosteroid. Illustrative barbituates include, but are not limited to allobarbital (5,5-diallylbarbiturate), amobarbital (5-ethyl-5-isopentyl-barbiturate), aprobarbital (5-allyl-5-isopropyl-barbiturate), alphenal (5-allyl-5-phenyl-barbiturate), barbital (5,5-diethylbarbiturate), brallobarbital (5-allyl-5-(2-bromo-allyl)-barbiturate), pentobarbital (5-ethyl-5-(l-methylbutyl)-barbiturate), phenobarbital (5-ethyl-5-phenylbarbiturate), secobarbital (5-[(2R)-pentan-2-yl]-5-prop-2-enyl-barbiturate), and the like. Illustrative benzodiazepines include, but are not limited to alprazolam, bromazepam, chlordiazepoxide, clonazepam, clorazepate, diazepam, estazolam, flurazepam, halazepam, ketazolam, lorazepam, nitrazepam, oxazepam, prazepam, quazepam, temazepam, triazolam, and the like. Illustrative neurosteroids include, but are not limited to allopregnanolone, and pregnanolone. The PAMs are preferably administered in a daily dose of 10% to 100%, preferably 10%-25%, of the Defined Daily Dose (DDD) as provided for the specific compound in the Anatomical Therapeutic Chemical (ATC) classification system maintained by the WHO Collaborating Centre for Drug Statistics Methodology (https://www.whocc.no/atc_ddd_index/). Furthermore, the 2-cyano-3-cyclopropyl hydroxy-n-aryl-thioacrylamide derivatives disclosed in WO2015140081 may be of use as PAMs in the present invention. These can be defined as compounds of the formula I or a tautomeric isoform thereof

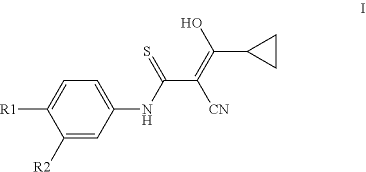

or a tautomeric isoform thereof, wherein R1 is selected from the group consisting of halogen, nitro, lower alkyl sulfonyl, cyano, trifluromethyl lower alkyl, lower alkoxy, lower alkoxycarbonyl, carboxy, lower alkyl aminosulfonyl, perfluoro lower alkyl, lower alkylthio, hydroxy lower alkyl, alkoxy lower alkyl, lower alkylthio lower alkyl, lower alkylsulfinyl lower alkyl, lower alkylsulfonyl lower alkyl, lower alkylsulfinyl, lower alkanoyl, aroyl, aryl, aryloxy, wherein the term “lower” as used above refers preferably to 1 to 3 carbon atoms, and R2 is selected from the group consisting of hydrogen, alkyl, alkoxy, alkylthio, and alkylcarbonyl, each alkyl residue having preferably 1 to 3 carbon atoms, and their tautomeric isoforms, non-toxic, pharmaceutically acceptable salts or pro-drugs thereof.

The term “hypoglycemia-associated autonomic failure”, or HAAF, is the failure of a subject to be aware of, and able to avoid, hypoglycemic events. It is considered to result from recent antecedent iatrogenic hypoglycemia which causes both defective glucose counterregulation by reducing the epinephrine response to falling glucose levels in the setting of an absent glucagon response, and hypoglycemia unawareness by reducing the autonomic and the resulting neurogenic symptom responses, producing a vicious cycle of recurrent hypoglycemia (Cryer, 2001).

The term “Selective Serotonin Reuptake Inhibitor” or SSRI, relates to a class of drugs that function by increasing the extracellular level of the neurotransmitter serotonin by limiting its reabsorption (reuptake) into the presynaptic cell, increasing the level of serotonin in the synaptic cleft available to bind to the postsynaptic receptor. Examples of SSRIs are citalopram, escitalopram, fluoxetine, fluvoxamine, paroxetine, and sertraline.

The term “prevent” is intended to encompass both total prevention and partial prevention, i.e. total or partial reduction of severity and/or likelihood of incidence of a prevented event or condition, as compared to a subject's historic pattern of the event and/or as compared to an untreated control. Assessment/scoring of severity of hypoglycemia is known in the art (Ryan, 2004).

### DETAILED DESCRIPTION OF THE INVENTION

A study according to Study Protocol 1 below was initiated under the study name Regenerate-1 (EudraCT Number: 2018-001115-73).

Surprisingly, it has been found that GABA can lower the pathologically increased circulating glucagon concentrations that are present in type 1 diabetes individuals to more normal concentrations. This capacity of GABA to decrease glucagon levels to normal is surprising in view of suggestions in the prior art, such as WO2019/114718, where GABA is suggested (but not shown) to acutely increase circulating glucagon levels by acting on glutamate receptors.

Glucagon concentrations are normally pathologically increased in both type 1 and type 2 diabetes individuals and these individuals thus have little or no capacity to further increase glucagon in response to hypoglycemia. It is important to address this deficiency in the counterregulatory hormone response (blood glucose increasing hormones) in order to protect these patients from severe hypoglycemia. The present invention aims to provide a solution to this clinical problem.

We have shown that by normalizing circulating glucagon levels at normoglycaemia, the capacity to increase glucagon at hypoglycemic attacks can be restored. This can be done through administration of GABA to individuals having higher than normal circulating glucagon levels at normoglycaemia.

Furthermore, individuals with type 1 or type 2 diabetes show a less than normal increase of other insulin counterregulatory hormones (growth hormone, cortisol and epinephrine (adrenalin)) in response to hypoglycemia, as compared to healthy individuals. The dose-escalation part of the study according to Study Protocol 1 shows that also the response of these hormones to hypoglycemia may be restored by GABA treatment. Without being bound by theory, it may be that normal physiological concentrations at normoglycemia are restored also in these cases.

The present invention also relates to combinations of GABA with other compounds that have a positive effect in methods for prevention, or reducing the risk of, hypoglycemia in a subject or that enhance the effect of GABA in such methods, or have therapeutic effects on their own that act in synergy with GABA.

Examples of compounds that enhance the effect of GABA include Positive Allosteric Modulators as defined above.

Examples of compounds that have therapeutic effects on their own and act in synergy with GABA include SSRIs, such as fluoxetine, and the endogenous hormone dehydroepiandrosterone.

Study Protocol 1

The following protocol can be used to investigate the effect of oral GABA therapy on hypoglycemia.

Objectives:

Primary Objective

The primary objective is to evaluate the acute and long-term safety of oral GABA treatment.

Secondary Objectives

The secondary objectives are to evaluate the different effect between the three treatment groups. As well as to analyze the outcome of oral GABA treatment, with or without combination with alprazolam treatment, on regaining endogenous insulin secretion as measured by C-peptide, overall diabetes status, serum levels of GABA, effects on the immune system and quality of life (QoL) of the patients (using the DTSQ and RAND-36 questionnaires).

Endpoints:

Primary Endpoint

The primary endpoints in this study is:
Number of AEs/SAEs possibly or probably related to GABA treatment or GABA treatment in combination with Alprazolam

Changes in laboratory parameters, physical examinations and vital signs over time vs baseline values.

Secondary Endpoints
Difference in C-peptide (Area Under the Curve [AUC]mean 0-120 min) during a Mixed Meal Tolerance Test (MMTT) between baseline and after 3 months of treatment, 6 months of treatment and the follow-up visit respectively for all treatments (Low dose daily oral GABA treatment, High dose daily oral GABA treatment, High dose oral GABA in combination with Alprazolam treatment).
Difference in C-peptide (Area Under the Curve [AUC]mean 0-120 min) during an MMTT between baseline and after 3 months of treatment, 6 months of treatment and the follow-up visit respectively for all treatments. Analyzed with an ultra-sensitive ELISA.
Difference in maximum stimulated C-peptide during an MMTT between baseline and after 3 months of treatment, 6 months of treatment and the follow-up visit respectively for all treatments.
Difference in maximum stimulated C-peptide during an MMTT between baseline and after 3 months of treatment, 6 months of treatment and the follow-up visit respectively for all treatments. Analyzed with an ultra-sensitive ELISA.
Difference between the treatments in difference in C-peptide (Area Under the Curve [AUC]mean 0-120 min) during an MMTT between baseline and after 3 months of treatment, 6 months of treatment and the follow-up visit.
Difference in glucagon (Area Under the Curve [AUC]mean 0-120 min) during an Hypoglycemic Clamp between baseline and 6 months of treatment for all treatments.
Difference between treatments in difference in glucagon (Area Under the Curve [AUC]mean 0-120 min) during an Hypoglycemic Clamp between baseline and 6 months of treatment.
Variables that indicate diabetes status such as plasma C-peptide, glucagon, proinsulin, proinsulin/C-peptide, glucagon like-peptide 1, lipids Hemoglobin A1c (HbA1c) and insulin adjusted HbA1c (IDAAC). Daily exogenous insulin consumption, variability of blood sugar, and number of self-reported hypoglycemia.
Variables that indicate effects on the immune system such as serum autoantibodies (and isotypes) to GAD65 and Islet Antigen-2 and a general profiling of the immune cells in the blood.
Analysis of GABA plasma levels after 3 and 6 months of treatment and at the follow-up visit.
Measurements of patient QoL by questionnaire.

Trial Design

Description of the Study Design and Procedures

Patients will provide written informed consent before any study-related procedures are performed. The first part of the study will include 6 patients and be performed as a Safety and Dose Escalation study in three steps, with a low (200 mg), medium (600 mg) and high dose (1200 mg) of GABA. Safety parameters, plasma concentration of GABA, insulin and glucose tolerance will then be evaluated. After the first patient has completed the three doses escalation steps a DSMB will review the safety data and if no safety concerns are arisen the other 5 patients will be allowed to start.

The treatment schedule is given below.

When all 6 patients have completed the dose-escalation study a DSMB will again review the safety data. If no safety concerns are arisen the subjects already included in this part of the study will then be enrolled in the main study, after a washout period of at least 1 month.

The main study is a 3-arm, open label, single center, clinical trial. Eligible patients will be randomized into one of three active treatment arms to receive oral GABA treatment for 6 months or oral GABA in combination with Alprazolam for 3 months, followed by treatment with GABA alone for 3 months (total treatment period 6 months).

Patients will be followed for a total of 7-9 months, depending on assigned treatment group. All patients will continue to receive their normal standard of care and intensive insulin treatment from their personal physicians during the whole study period.

On Day 1 (Visit 2), 2-4 weeks after the Screening Visit (the patients that were included in the dose-escalation study will not do a screening visit but will have a washout period of at least 1 month), patients eligible for the study will be randomized in a 1:1:1 ratio stratified by the C-peptide level to receive:
1) 200 mg of Remygen for 6 months (from Day 1 to Day 181),
2) 600 mg of Remygen for 6 months (from Day 44 to Day 225)
3) 600 mg of Remygen for 6 months (Day 44 to Day 225) and 0.5 mg Alprazolam for 3 months (Day 44 to Day 134)

The treatment schedule for Arm 1, Arm 2 and Arm 3 is described in Table 2, Table 3 and Table 4 respectively.

At the screening visit patients will be assigned a 3-digit sequential screening number, which will be used as a 3-digit patient identification (Patient Number), i.e. XXX.

Individual patients will be exposed to Remygen for 6 months and to Alprazolam for 3 months. All patients will continue to receive intensive insulin treatment from their personal physicians during the whole study period.

TABLE 1
Schedule of Patient Visits for safety and Dose Escalation Study period

| Safety and Dose Escalation study | | | | | | | | | |
|---|---|---|---|---|---|---|---|---|---|
| Screening | | | | | | | | | |
| Day −14 | | | | | | | | | |
| to −28 | Baseline | | Intervention | | | | | | |
| Screening | Day 1 | Day 2 | Day 3 | Day 5 | Day 6 | Day 8 | Day 9 | Day 10 | Day 11* |
| Visit no: | | | | | | | | | |
| 1 | 2 | 3 | 4 | 5 | 6 | 7 | 8 | 9 | 10 |

| | | | | | | | | | |
|---|---|---|---|---|---|---|---|---|---|
| 6subjects | MMTT | Hypogly- | Treatment | Safety, | Dose | Safety, | Dose | Safety, | Safety, |
| Baseline | cemic | start, | Hypogly- | escalation, | Hypogly- | escalation, | Hypogly- | MMTT | |
| samples | clamp | PK curve | cemic | Safety, | cemic | Safety, | cemic | | |
| | | | clamp | PK curve | clamp | PK curve | clamp | | |

| | | | |
|---|---|---|---|
| Dose/day | 200 mg of GABA | 600 mg GABA | 1200 mg of GABA |

*A DSMB will evaluate the first patient and decide if the other 5 patients can enter the study. After all 6 patients have completed the dose escalation steps the DSMB will again review the safety data and decide if the main study can start.

TABLE 2
Schedule of Patient Visits for Main Study Period - Arm 1

| GABA Study | | | | | | | | | |
|---|---|---|---|---|---|---|---|---|---|
| Screening | Intervention | | | | | | | | |
| Day −14 | Day 1 and 2 | | | | | | | Follow-up | Follow-up |
| to −28 | BASELINE VISIT & | | | | Day 90 | Day 180 and 181 | | Day 210 | Day 211 |
| Screening | Randomization | | Day 7 | Day 30* | M 3 | M 6 | | M 7 | M 7 |
| Visit no: | | | | | | | | | |
| 1 | 2 | 3 | 4 | 5 | 6 | 7 | 8 | 9 | 10 |

| | | | | | | | | |
|---|---|---|---|---|---|---|---|---|
| Group 1 | MMTT, | Hypogly- | Safety | MMTT, | MMTT, | Hypogly- | MMTT | Hypogly- |
| (12 | Baseline | cemic | | Safety | Safety | cemic | | cemic |
| subjects) | samples | Clamp | | | | clamp | | clamp |

| | |
|---|---|
| Dose/day | Low dose of GABA (200 mg/day) |

*DSMB will evaluate the safety data of the first 4 patients included.

TABLE 3
Schedule of Patient Visits for Main Study Period - Arm 2

| GABA Study | | | | | | | | | | |
|---|---|---|---|---|---|---|---|---|---|---|
| Screening | Intervention | | | | | | | | | |
| Day −14 | Day 1 and 2 | | | | | | | | Follow-up | Follow-up |
| to −28 | BASELINE VISIT & | | Day 44 | Day 51 | Day 74 | Day 134 | Day 224 and 225 | | Day 254 | Day 255 |
| Screening | Randomization | | Month 2* | M 2 | M 3# | M 5 | M 8 | | M 9 | M 9 |
| Visit no: | | | | | | | | | | |
| 1 | 2 | 3 | 4 | 5 | 6 | 7 | 8 | 9 | 10 | 11 |

| | | | | | | | | | | |
|---|---|---|---|---|---|---|---|---|---|---|
| Group 2 | Z | MMTT, | Hypogly- | Treatment | Safety | MMTT, | MMTT, | Hypogly- | MMTT | Hypogly- |
| (12 | | Baseline | cemic | start, | | Safety | Safety | cemic | | cemic |
| subjects) | | samples | clamp | MMTT | | | | clamp | | clamp |

| | |
|---|---|
| Dose/day | High dose of GABA (600 mg/day) |

*DSMB will evaluate the safety data of the first 4 patients included and decide if the second treatment arm can start. Therefore, start of treatment for treatment arm 2 will be delayed.
#The DSMB will evaluate the safety data of the first 4 patients included in treatment arm 2 after 1 month of treatment to make sure that no safety concerns have arisen upon treatment with the higher dose of GABA.

TABLE 4
Schedule of Patient Visits for Main Study Period - Arm 3

| GABA Study | | | | | | | | | | |
|---|---|---|---|---|---|---|---|---|---|---|
| Screening | Intervention | | | | | | | | | |
| Day −14 | Day 1 and 2 | | | | | | | | Follow-up | Follow-up |
| to −28 | BASELINE VISIT & | | Day 44 | Day 51 | Day 74 | Day 134 | Day 224 and 225 | | Day 254 | Day 255 |
| Screening | Randomization | | Month 2*£ | M 2 | M 3# | M5$ | M 8 | | M 9 | M 9 |
| Visit no: | | | | | | | | | | |
| 1 | 2 | 3 | 4 | 5 | 6 | 7 | 8 | 9 | 10 | 11 |

| | | | | | | | | | |
|---|---|---|---|---|---|---|---|---|---|
| Group 3 | MMTT, | Hypogly- | Treatment | Safety | MMTT, | MMTT, | Hypogly- | MMTT | Hypogly- |
| (12 | Baseline | cemic | start, | | Safety | Safety | cemic | | cemic |
| subjects) | samples | clamp | MMTT | | | | clamp | | clamp |

| | | |
|---|---|---|
| Dose/day | High dose of GABA (600 mg/day) + | High dose of GABA |
| | 0.5 mg/day Alprazolam | (600 mg/day) |

*DSMB will evaluate the safety data of the first 4 patients included in treatment arm 1 and decide if treatment arm 3 can start. Therefore, start of treatment for treatment arm 3 will be delayed.
£Treatment will be initiated on day 44 with 0.5 mg of Alprazolam. Treatment will be ramped up on day 45 with Low dose GABA (200 mg) and 0.5 mg Alprazolam. On day 46, High dose GABA (600 mg) and 0.5 mg Alprazolam will be administered onwards for the remaining treatment period. Patients will remain under observation at the hospital for 3 h after tablet intake days 44-46 as an extra precaution.
#The DSMB will evaluate the safety data of the first 4 patients included in treatment arm 3 after 1 month of treatment to make sure that no safety concerns have arisen upon treatment with the higher dose of GABA + Alprazolam.
$Treatment will Alprazolam will stop after 3 months and patients will continue with only oral GABA for 3 months.

TABLE 5
Schedule of Events for Safety and Dose Escalation Study

| | Event | | | | | | | | | |
|---|---|---|---|---|---|---|---|---|---|---|
| | | V2 | V3 | | | | | | | |
| | V1 | Baseline | Baseline | | | | | | | |
| | Screening | Visit | Visit | V4 | V5 | V6 | V7 | V8 | V9 | V10 |
| | DAY | | | | | | | | | |
| | −14 to −28 | 1 | 2 | 3 | 5 | 6 | 8 | 9 | 10 | 11 |
| Informed Consent | X | | | | | | | | | |
| Eligibility check | X | X | | | | | | | | |
| Demographics | X | | | | | | | | | |
| Medical History | X | | | | | | | | | |
| Family history of T1D | X | | | | | | | | | |
| GABA start/end | | | | X | | | | | | X |
| Dose escalation | | | | | | X | | X | | |
| General Physical | X | X | | | | X | | X | | X |
| Exam | | | | | | | | | | |
| Neurological | X | X | | | | X | | X | | X |
| Assessment | | | | | | | | | | |
| ECG | X | X | | | | X | | X | | X |
| Concomitant | X | X | | | | | | | | |
| Medication | | | | | | | | | | |
| Vital signs (BP) | X | X | | | | X | | X | | X |
| AEs | | | | | X | X | X | X | X | X |
| Self-reported severe | | X | | | | X | | X | | X |
| hypoglycemia | | | | | | | | | | |
| MMTT | | X | | | | | | | | X |
| Hypoglycemic | | | X | | X | | X | | X | |
| clamp | | | | | | | | | | |
| FreeStyle LibrePro, | X | X | | | | | | | | X |
| FGM, start/end | | | | | | | | | | |

Blood Sampling for Safety, GABA levels and Immunology:

| | | | | | | | | |
|---|---|---|---|---|---|---|---|---|
| Hematology | X | X | | X | | X | | X |
| Clinical Chemistry | X | X | | X | | X | | X |
| GABA level | | X | | | | | | X |
| GABA PK Curve | | | X | | X | | X | |
| Immunological | | X | | | | | | X |
| parameters | | | | | | | | |

Urine Analysis:

| | | | | | | | | |
|---|---|---|---|---|---|---|---|---|
| Microalbuminuria | X | X | | X | X | X | X | X |
| Creatinine | X | X | | X | X | X | X | X |
| GABA | | X | X | | X | | X | |

Blood Sampling for Diabetes Status:

| | | | | | | | | | |
|---|---|---|---|---|---|---|---|---|---|
| Fasting C-peptide | X | X | | | | | | | X |
| MMTT-induced | | X | | | | | | | X |
| C-peptide | | | | | | | | | |
| HbA1c | X | X | | | | | | | |
| Fasting Glucose | X | X | | X | X | X | X | | X |
| MMTT-induced | | X | | | | | | | X |
| Glucose | | | | | | | | | |
| Clamp-induced | | | X | X | | X | | X | |
| glucagon, | | | | | | | | | |
| epinephrine, | | | | | | | | | |
| norepinephrine, | | | | | | | | | |
| growth hormone | | | | | | | | | |
| and cortisol | | | | | | | | | |
| Clamp-induced | | | X | X | | X | | X | |
| Glucose | | | | | | | | | |

Abbreviations:
AEs = Adverse Events;
BP = Blood Pressure;
HBA1c = Hemoglobin A1c;
MMTT = mixed meal tolerance test;
ITT = Insulin Tolerance Test;
FGM = Flash Glucose Monitoring;
T1D = Type 1 Diabetes;
PK = Pharmacokinetic

TABLE 6
Schedule of Events for Main Study period - Treatment arm 1

| | Event | | | | | | | | | |
|---|---|---|---|---|---|---|---|---|---|---|
| | | V2 | V3 | V4 | V5 | V6 | V7 | V8 | V9 | V10 |
| | V1 | Baseline | Baseline | Month | Month | Month | Month | Month | Month | Month |
| | Screening | Visit | Visit | 1 | 1 | 3 | 6 | 6 | 7 | 7 |
| | DAY | | | | | | | | | |
| | −14 | | | 7 | 30 | 90 | 180 | 181 | 210 | 211 |
| | to −28 | 1 | 2 | (±3) | (±5) | (±5) | (±5) | (±5) | (±14) | (±14) |
| Informed Consent | X | | | | | | | | | |
| Eligibility check | X | X | | | | | | | | |
| Demographics | X | | | | | | | | | |
| Randomization | | X | | | | | | | | |
| Medical History | X | | | | | | | | | |
| Family history of T1D | X | | | | | | | | | |
| GABA start/end | | | X | | | | | X | | |
| General Physical Exam | X | X | | X | X | X | X | | X | |
| Neurological Assessment | X | X | | X | X | X | X | | X | |
| ECG | X | | | | X | | | | X | |
| Concomitant Medication | X | X | | X | X | X | X | | X | |
| Vital signs (BP) | X | X | | X | X | X | X | | | |
| Blood glucose and Insulin | | X | | X | X | X | X | | X | |
| dose diary collected | | | | | | | | | | |
| Freestyle LibrePro, FGM | X | | | | | X | X | | X | |
| AEs | | | | X | X | X | X | | X | |
| Self-reported severe | | X | | X | X | X | X | | X | |
| hypoglycemia | | | | | | | | | | |
| QoL questionnaire | | X | | | | | X | | X | |
| MMTT | | X | | | | X | X | | X | |
| Hypoglycemic Clamp | | | X | | | | | X | | X |
| Stool samples | | X | | | | X | X | | X | |
| Hand out patient blood | X | X | | X | X | X | X | | | |
| glucose and insulin diary | | | | | | | | | | |

Blood Sampling for Safety, GABA levels and Immunology:

| | | | | | | | |
|---|---|---|---|---|---|---|---|
| Hematology | X | X | X | X | X | X | X |
| Clinical Chemistry | X | X | X | X | X | X | X |
| GABA level | | X | X | X | X | X | X |
| PBMC and/or | | X | | X | X | X | X |
| Immunological parameters | | | | | | | |

Urine Analysis:

| | | | | | | | |
|---|---|---|---|---|---|---|---|
| Microalbuminuria | X | X | X | X | X | X | X |
| Creatinine | X | X | X | X | X | X | X |

Blood Sampling for Diabetes Status:

| | | | | | | | | | | |
|---|---|---|---|---|---|---|---|---|---|---|
| Fasting C-peptide | X | X | | | X | X | X | | X | |
| MMTT-induced C-peptide, | | X | | | | X | X | | X | |
| insulin and proinsulin | | | | | | | | | | |
| HbA1c | X | X | | | X | X | X | | X | |
| Fasting Glucose | X | X | | X | X | X | X | | X | |
| MMTT-induced Glucose | | X | | | | X | X | | X | |
| Clamp-induced glucagon, | | | X | | | | | X | | X |
| epinephrine, | | | | | | | | | | |
| norepinephrine, growth | | | | | | | | | | |
| hormone and cortisol | | | | | | | | | | |
| Clamp-induced Glucose | | | X | | | | | X | | X |
| Glucagon, GLP-1, lipids | | X | | | X | X | X | | X | |

Abbreviations:
AEs = Adverse Events;
BP = Blood Pressure;
HBA1c = Hemoglobin A1c;
MMTT = mixed meal tolerance test;
ITT = Insulin Tolerance Test;
FGM = Flash Glucose Monitoring;
T1D = Type 1 Diabetes;
QoL = Quality of Life;
PK = Pharmacokinetic

TABLE 7
Schedule of Events for Main Study period - Treatment arm 2

| | Event | | | | | | | | | | |
|---|---|---|---|---|---|---|---|---|---|---|---|
| | | V2 | V3 | V4 | V5 | V6 | V7 | V8 | V9 | V10 | V11 |
| | V1 | Baseline | Baseline | Month | Month | Month | Month | Month | Month | Month | Month |
| | Screening | Visit | Visit | 2 | 2 | 3 | 5 | 8 | 8 | 9 | 9 |
| | DAY | | | | | | | | | | |
| | −14 | | | 44 | 51 | 74 | 134 | 224 | 225 | 254 | 255 |
| | to −28 | 1 | 2 | (±3) | (±5) | (±5) | (±5) | (±5) | (±5) | (±14) | (±14) |
| Informed Consent | X | | | | | | | | | | |
| Eligibility check | X | X | | | | | | | | | |
| Demographics | X | | | | | | | | | | |
| Randomization | | X | | | | | | | | | |
| Medical History | X | | | | | | | | | | |
| Family history of T1D | X | | | | | | | | | | |
| GABA start/end | | | | X | | | | | X | | |
| General Physical Exam | X | X | | X | X | X | X | X | | X | |
| Neurological | X | X | | X | X | X | X | X | | X | |
| Assessment | | | | | | | | | | | |
| ECG | X | | | | | X | | | | X | |
| Concomitant | X | X | | X | X | X | X | X | | X | |
| Medication | | | | | | | | | | | |
| Vital signs (BP) | X | X | | X | X | X | X | X | | | |
| Blood glucose and | | X | | X | X | X | X | X | | X | |
| insulin dose diary | | | | | | | | | | | |
| collected | | | | | | | | | | | |
| Freestyle LibrePro, | X | | | | | | X | X | | X | |
| FGM | | | | | | | | | | | |
| AEs | | | | | X | X | X | X | | X | |
| Self-reported severe | | X | | X | X | X | X | X | | X | |
| hypoglycemia | | | | | | | | | | | |
| QoL questionnaire | | X | | X | | | | X | | X | |
| MMTT | | X | | X | | | X | X | | X | |
| Hypoglycemic Clamp | | | X | | | | | | X | | X |
| Stool samples | | X | | X | | | X | X | | X | |
| Hand out patient blood | X | X | | X | X | X | X | X | | | |
| glucose and insulin | | | | | | | | | | | |
| diary | | | | | | | | | | | |

Blood Sampling for Safety, GABA levels and Immunology:

| | | | | | | | | |
|---|---|---|---|---|---|---|---|---|
| Hematology | X | X | X | X | X | X | X | X |
| Clinical Chemistry | X | X | X | X | X | X | X | X |
| GABA level | | X | X | X | X | X | X | X |
| PBMC and/or | | X | | | X | X | X | X |
| Immunological | | | | | | | | |
| parameters | | | | | | | | |

Urine Analysis:

| | | | | | | | | |
|---|---|---|---|---|---|---|---|---|
| Microalbuminuria | X | X | X | X | X | X | X | X |
| Creatinine | X | X | X | X | X | X | X | X |

Blood Sampling for Diabetes Status:

| | | | | | | | | | | | |
|---|---|---|---|---|---|---|---|---|---|---|---|
| Fasting C-peptide, | X | X | | X | | X | X | X | | X | |
| MMTT-induced | | X | | X | | | X | X | | X | |
| C-peptide, insulin and | | | | | | | | | | | |
| proinsulin | | | | | | | | | | | |
| HbA1c | X | X | | X | | X | X | X | | X | |
| Fasting Glucose | X | X | | X | X | X | X | X | | X | |
| MMTT-induced Glucose | | X | | X | | | X | X | | X | |
| Clamp-induced | | | X | | | | | | X | | X |
| glucagon epinephrine, | | | | | | | | | | | |
| norepinephrine, growth | | | | | | | | | | | |
| hormone and cortisol | | | | | | | | | | | |
| Clamp-induced Glucose | | | X | | | | | | X | | X |
| Glucagon, GLP-1, | | X | | X | | X | X | X | | X | |
| lipids | | | | | | | | | | | |

Abbreviations:
AEs = Adverse Events;
BP = Blood Pressure;
HBA1c = Hemoglobin A1c;
MMTT = mixed meal tolerance test;
ITT = Insulin Tolerance Test;
FGM = Flash Glucose Monitoring;
T1D = Type 1 Diabetes;
QoL = Quality of Life;
PK = Pharmacokinetic

TABLE 8
Schedule of Events for Main Study period - Treatment arm 3

| | Event | | | | | | | | | | |
|---|---|---|---|---|---|---|---|---|---|---|---|
| | | V2 | V3 | V4 | V5 | V6 | V7 | V8 | V9 | V10 | V11 |
| | V1 | Baseline | Baseline | Month | Month | Month | Month | Month | Month | Month | Month |
| | Screening | Visit | Visit | 2 | 2 | 3 | 5 | 8 | 8 | 9 | 9 |
| | DAY | | | | | | | | | | |
| | −14 | | | 44 | 51 | 74 | 134 | 224 | 225 | 254 | 255 |
| | to −28 | 1 | 2 | (±3) | (±5) | (±5) | (±5) | (±5) | (±5) | (±14) | (±14) |
| Informed Consent | X | | | | | | | | | | |
| Eligibility check | X | X | | | | | | | | | |
| Demographics | X | | | | | | | | | | |
| Randomization | | X | | | | | | | | | |
| Medical History | X | | | | | | | | | | |
| Family history of T1D | X | | | | | | | | | | |
| GABA start/end | | | | X | | | | | X | | |
| Alprazolam start/end | | | | X | | | X | | | | |
| General Physical Exam | X | X | | X | X | X | X | X | | X | |
| Neurological | X | X | | X | X | X | X | X | | X | |
| Assessment | | | | | | | | | | | |
| ECG | X | | | | | X | | | | X | |
| Concomitant | X | X | | X | X | X | X | X | | X | |
| Medication | | | | | | | | | | | |
| Vital signs (BP) | X | X | | X | X | X | X | X | | | |
| Blood glucose and | | X | | X | X | X | X | X | | X | |
| insulin dose diary | | | | | | | | | | | |
| collected | | | | | | | | | | | |
| FreeStyle LibrePro, | X | | | | | | X | X | | X | |
| FGM | | | | | | | | | | | |
| AEs | | | | | X | X | X | X | | X | |
| Self-reported severe | | X | | X | X | X | X | X | | X | |
| hypoglycemia | | | | | | | | | | | |
| QoL questionnaire | | X | | X | | | | X | | X | |
| MMTT | | X | | X | | | X | X | | X | |
| Hypoglycemic Clamp | | | X | | | | | | X | | X |
| Stool samples | | X | | X | | | X | X | | X | |
| Hand out patient blood | X | X | | X | X | X | X | X | | | |
| glucose and insulin | | | | | | | | | | | |
| diary | | | | | | | | | | | |

Blood Sampling for Safety, GABA levels and Immunology:

| | | | | | | | | |
|---|---|---|---|---|---|---|---|---|
| Hematology | X | X | X | X | X | X | X | X |
| Clinical Chemistry | X | X | X | X | X | X | X | X |
| GABA level | | X | X | X | X | X | X | X |
| PBMC and/or | | X | | | X | X | X | X |
| Immunological | | | | | | | | |
| parameters | | | | | | | | |

Urine Analysis:

| | | | | | | | | |
|---|---|---|---|---|---|---|---|---|
| Microalbuminuria | X | X | X | X | X | X | X | X |
| Creatinine | X | X | X | X | X | X | X | X |

Blood Sampling for Diabetes Status:

| | | | | | | | | | | | |
|---|---|---|---|---|---|---|---|---|---|---|---|
| Fasting C-peptide, | X | X | | X | | X | X | X | | X | |
| MMTT-induced | | X | | X | | | X | X | | X | |
| C-peptide, insulin and | | | | | | | | | | | |
| proinsulin | | | | | | | | | | | |
| HbA1c | X | X | | X | | X | X | X | | X | |
| Fasting Glucose | X | X | | X | X | X | X | X | | X | |
| MMTT-induced Glucose | | X | | X | | | X | X | | X | |
| Clamp-induced | | | X | | | | | | X | | X |
| glucagon, epinephrine, | | | | | | | | | | | |
| norepinephrine, growth | | | | | | | | | | | |
| hormone and cortisol | | | | | | | | | | | |
| Clamp-induced Glucose | | | X | | | | | | X | | X |
| Glucagon, GLP-1, | | X | | X | | X | X | X | | X | |
| lipids | | | | | | | | | | | |

Abbreviations:
AEs = Adverse Events;
BP = Blood Pressure;
HBA1c = Hemoglobin A1c;
MMTT = mixed meal tolerance test;
ITT = Insulin Tolerance Test;
FGM = Flash Glucose Monitoring;
T1D = Type 1 Diabetes;
QoL = Quality of Life;
PK = Pharmacokinetic

Study Procedures

The study procedures and assessments are described in the section below and presented in detail in Section Safety, Diabetes status assessments, Immunological assessments, GABA levels in plasma, Stool samples, Quality of Life, Concomitant medication assessments, Demographics and Blood sampling procedures. Recording and reporting of AEs are also described below.

The timing of all study events is shown in Table 5-8 and below.

All Visits

Note that the patient should attend all study visits in the morning following an overnight fast (>10 hours, water allowed) and without taking their daily dose of Remygen and Alprazolam. Patients will be instructed during the visits when to take their daily dose of Remygen and Alprazolam that specific day. For patients with evidence of an infection (including fever), the complete visit should be postponed for 5 days or until the patient has recovered.

Timing of all visits, Visit 1 through Visit 10 (arm 1)/Visit 11 (arms 2 and 3)

The first visit, the screening visit (Visit 1) should be performed 14 to 28 days before planned Visit 2 (Baseline) for both the Safety and Dose Escalation study and the Main study. For the Safety and Dose Escalation study, enrolled patients must be able to come to the site on the specified days. There is no visit window allowed for this part of the study.

For the main study, visits should be scheduled within +/−3 days for visit 4, +/−5 days for Visit 5 to Visit 8 (arm 1)/9 (arms 2 and 3) and within +/−14 days for Visit 9 and 10 (arm 1)/10 and 11 (arms 2 and 3).

DSMB will evaluate the safety data of the first 4 patients included in arm 1 after one month of GABA treatment and decide if treatment in arms 2 and 3 can be started. Visit 4 (treatment start) in arms 2 and 3 may therefore in first patients be delayed, but will be scheduled within 14 days after such approval.

Description of Visits—Safety and Dose Escalation

Visit S1, Screening (Day −14 to −28)

Potential study patients will receive both written and oral information about the study procedures, potential risks, and benefits. Before any study specific procedures may be performed, the patients must sign the informed consent form (ICF). After written informed consent has been obtained, the following activities and assessments will be performed and any SAEs occurring after signing the ICF will be recorded:
Eligibility will be checked versus the inclusion/exclusion criteria
Demographics (including age and gender)
Medical history
Family history of T1D
General Physical Examination, including weight and height
Neurological assessment
ECG
Concomitant medication
Vital signs (Blood Pressure [BP])
Blood sampling for hematology and clinical chemistry
Urine analysis for microalbuminuria and creatinine
Blood samples for diabetes status (fasting C-peptide, HbA1c and fasting glucose)
Glycemic variability/fluctuations device (FreeStyle LibrePro, FGM) will be given to the patient

Examinations will be performed according to Table 5.

Visit 52, Baseline (Day 1)

At Visit S2 (Baseline, Day 1) the following activities and assessments will be performed:
Eligibility will be checked versus the inclusion/exclusion criteria
General Physical Examination, including weight
Neurological assessment
ECG
Concomitant medication
Vital signs (BP)
Blood sampling for hematology, clinical chemistry, GABA levels, and immunological analyses
Urine analysis for microalbuminuria, creatinine and GABA
Self-reported severe hypoglycemia (defined as needing help from others and/or seizures and/or unconsciousness)
Mixed Meal Tolerance Test (MMTT)
Blood samples for diabetes status (fasting C-peptide, MMTT-induced C-peptide, HbA1c, fasting glucose and MMTT-induced glucose)
Glycemic variability/fluctuations device (FreeStyle LibrePro, FGM) will be given to the patient

Examinations will be performed according to Table 5.

Visit 53, Baseline (Day 2)

At Visit S3 (Baseline, Day 2) the following activities and assessments will be performed:
Hypoglycemic clamp
Blood samples for diabetes status (Clamp-induced glucagon, epinephrine, norepinephrine, growth hormone, cortisol and glucose)

Examinations will be performed according to Table 5.

Visit 54, (Day 3)

At Visit S4 (Day 3)
Treatment start with GABA (lowest dose)
Blood sampling for Pharmacokinetic evaluation of GABA
Urine analysis of GABA

Examinations will be performed according to Table 5.

Visit 55 (Day 5)

At Visit S5 (Day 5) the following activities and assessments will be performed:
AEs
Blood sampling for hematology, clinical chemistry and immunological analyses
Urine analysis for microalbuminuria and creatinine
Hypoglycemic clamp
Blood samples for diabetes status (fasting glucose, clamp-induced glucagon, epinephrine, norepinephrine, growth hormone, cortisol and glucose)

Examinations will be performed according to Table 5.

Visit 56 (Day 6)

At Visit S6 (Day 6) the following activities and assessments will be performed:
General Physical Examination
Neurological assessment
ECG
Vital signs (BP)
AEs
Urine analysis for microalbuminuria, creatinine and GABA
Self-reported severe hypoglycemia (defined as needing help from others and/or seizures and/or unconsciousness)
Dose escalation of GABA (medium dose)
Blood sampling for Pharmacokinetic evaluation of GABA

Examinations will be performed according to Table 5

Visit 57 (Day 8)

At Visit S7 (Day 8) the following activities and assessments will be performed:
AEs
Blood sampling for hematology, clinical chemistry, and immunological analyses
Urine analysis for microalbuminuria and creatinine
Hypoglycemic clamp
Blood samples for diabetes status (fasting glucose, clamp-induced glucagon, epinephrine, norepinephrine, growth hormone, cortisol and glucose)

Examinations will be performed according to Table 5

Visit 58 (Day 9)

At Visit S8 (Day 9) the following activities and assessments will be performed:

General Physical Examination
Neurological assessment
ECG
Vital signs (BP)
AEs
Urine analysis for microalbuminuria, creatinine and GABA
Self-reported severe hypoglycemia (defined as needing help from others and/or seizures and/or unconsciousness)
Dose escalation of GABA (Highest dose)
Blood sampling for Pharmacokinetic evaluation of GABA

Examinations will be performed according to Table 5

Visit 59 (Day 10)

At Visit S9 (Day 10) the following activities and assessments will be performed:
AEs
Hypoglycemic clamp
Blood samples for diabetes status (clamp-induced glucagon, epinephrine, norepinephrine, growth hormone, cortisol and glucose.

Examinations will be performed according to Table 5

Visit 510 (Day 11)

At Visit S10 (Day 11) the following activities and assessments will be performed:
General Physical Examination, including weight
Neurological assessment
ECG
Vital signs (BP)
AEs
Blood sampling for hematology, clinical chemistry, GABA levels, and immunological analyses
Urine analysis for microalbuminuria and creatinine
Self-reported severe hypoglycemia (defined as needing help from others and/or seizures and/or unconsciousness)
Mixed Meal Tolerance Test (MMTT)
Blood samples for diabetes status (fasting C-peptide, MMTT-induced C-peptide, HbA1c, fasting glucose and MMTT-induced glucose)
Glycemic variability/fluctuations device (FreeStyle LibrePro, FGM) is returned by the patient

Examinations will be performed according to Table 5

One patient will first perform all visits. A DSMB will then review the safety data, if there are no safety concerns the other 5 patients that are enrolled will be allowed to start the study.

A DSMB will then review the safety data again from the Safety and Dose Escalation study and decide if the Main Study can start.

Description of Visits—Main Study, Treatment Arm 1

Visit 1, Screening (Day −14 to −28)

Patients that were included in the Safety and Dose Escalation study will be offered to take part in the main study and will not do the screening visit but will have a washout period for at least 1 month. Potential study patients will receive both written and oral information about the study procedures, potential risks, and benefits. Before any study specific procedures may be performed, the patients must sign the informed consent form (ICF). After written informed consent has been obtained, the following activities and assessments will be performed and any SAES occurring after signing the ICF will be recorded:
Eligibility will be checked versus the inclusion/exclusion criteria
Demographics (including age and gender)
Medical history
Family history of T1D
General Physical Examination, including weight and height
Neurological assessment
ECG
Concomitant medication
Vital signs (Blood Pressure [BP])
Blood sampling for hematology and clinical chemistry
Urine analysis for microalbuminuria and creatinine
Blood samples for diabetes status (fasting C-peptide, HbA1c and fasting glucose)
Glycemic variability/fluctuations device (Freestyle LibrePro, FGM) will be given to the patient
The patient will be given a patient diary and instructed to collect their blood glucose and insulin dose for 3 days prior to the next visit

Examinations will be performed according to Table 6

Visit Z Baseline (Day 1)

At Visit 2 (Baseline, Day 1) the following activities and assessments will be performed:
Eligibility will be checked versus the inclusion/exclusion criteria
Randomization into treatment arm 1, 2 or 3
General Physical Examination, including weight
Neurological assessment
Concomitant medication
Vital signs (BP)
Blood sampling for hematology, clinical chemistry, GABA levels and immunological analyses and PBMC isolation
Urine analysis for microalbuminuria and creatinine
Patient will be asked to provide stool samples
Mixed Meal Tolerance Test (MMTT)
Blood samples for diabetes status (fasting C-peptide, MMTT-induced C-peptide, HbA1c, fasting glucose and MMTT-induced glucose, insulin, proinsulin, glucagon, GLP-1 and lipids)
QoL questionnaire
Blood glucose and insulin dose collected (Exogenous insulin dose/kg/24 hours) for 3 days prior to visit
Self-reported severe hypoglycemia (defined as needing help from others and/or seizures and/or unconsciousness)

Examinations will be performed according to Table 6

Visit 3, Baseline (Day 2)

At Visit 3 (Baseline, Day 2) the following activities and assessments will be performed:

Hyperglycemic clamp
Blood samples for diabetes status (clamp-induced glucagon, epinephrine, norepinephrine, growth hormone, cortisol and glucose)
After the clamp is performed treatment with GABA will start. Remind the patient that the daily dose of GABA should be taken with food in the morning, except for days when they come in for a study visit.

Examinations will be performed according to Table 6

Visit 4 (Day 7)

At Visit 4 (Day 7) the following activities and assessments will be performed:
General Physical Examination, including weight
Neurological assessment
Concomitant medication
Vital signs (BP)
AEs
Blood sampling for hematology, clinical chemistry, GABA levels and fasting glucose
Urine analysis for microalbuminuria and creatinine
Blood glucose and insulin dose collected (Exogenous insulin dose/kg/24 hours) for 3 days prior to visit
Self-reported severe hypoglycemia (defined as needing help from others and/or seizures and/or unconsciousness)

Visit 5 (Day 30)

At Visit 4 (Day 7) and Visit 5 (Day 30) the following activities and assessments will be performed:
General Physical Examination, including weight
Neurological assessment
ECG
Concomitant medication
Vital signs (BP)
AEs
Blood sampling for hematology, clinical chemistry, GABA levels and immunological analyses
Urine analysis for microalbuminuria and creatinine
Blood glucose and insulin dose collected (Exogenous insulin dose/kg/24 hours) for 3 days prior to visit
Blood samples for diabetes status (fasting C-peptide, HbA1c, fasting glucose, glucagon, GLP-1 and lipids)
Self-reported severe hypoglycemia (defined as needing help from others and/or seizures and/or unconsciousness)

Examinations will be performed according to Table 6

After Visit 5 a DSMB will evaluate the safety data from the first 4 patients completing 1 month of treatment with GABA and evaluate if treatment arm 2 and 3 can start.

Visit 6 and 7 (Day 90 and 180)

At Visit 6 (Day 90) and Visit 7 (Day 180) the following activities and assessments will be performed:
General Physical Examination, including weight
Neurological assessment
Concomitant medication
Vital signs (BP)
AEs
Blood sampling for hematology, clinical chemistry, GABA levels, immunological analyses and PBMC isolation (PBMCs will only be collected at visit 7)
Urine analysis for microalbuminuria and creatinine
Patient will be asked to provide stool samples
Mixed Meal Tolerance Test (MMTT)
Blood samples for diabetes status (fasting C-peptide, MMTT-induced C-peptide, HbA1c, fasting glucose and MMTT-induced glucose, insulin, proinsulin, glucagon, GLP-1 and lipids)
QoL questionnaire (Visit 7)
Blood glucose and insulin dose collected (Exogenous insulin dose/kg/24 hours) for 3 days prior to visit
Glycemic variability/fluctuations device (Freestyle LibrePro, FGM) will be given to the patient (Visit 7)
Self-reported severe hypoglycemia (defined as needing help from others and/or seizures and/or unconsciousness)

Examinations will be performed according to Table 6

Visit 8 (Day 181)

At Visit 8 (Day 181) the following activities and assessments will be performed:
Hypoglycemic clamp
Blood samples for diabetes status (clamp-induced glucagon, epinephrine, norepinephrine, growth hormone, cortisol and glucose)
Treatment with GABA will end.

Examinations will be performed according to Table 6

Visit 9, Follow Up (Day 210)

At Visit 9 (Follow Up, Day 210) the following activities and assessments will be performed:
General Physical Examination, including weight
Neurological assessment
ECG
Concomitant medication
AEs
Blood sampling for hematology, clinical chemistry, GABA levels and immunological analyses and PBMC isolation
Urine analysis for microalbuminuria and creatinine
Patient will be asked to provide stool samples
Mixed Meal Tolerance Test (MMTT)
Blood samples for diabetes status (fasting C-peptide, MMTT-induced C-peptide, HbA1c, fasting glucose and MMTT-induced glucose, insulin, proinsulin, glucagon, GLP-1 and lipids)
QoL questionnaire
Blood glucose and insulin dose collected (Exogenous insulin dose/kg/24 hours) for 5 days prior to visit
Glycemic variability/fluctuations device (Freestyle LibrePro, FGM) will be returned by the patient
Self-reported severe hypoglycemia (defined as needing help from others and/or seizures and/or unconsciousness)

Examinations will be performed according to Table 6

Visit 10, Follow Up (Day 211)

At Visit 10 (Follow Up, Day 211) the following activities and assessments will be performed:
Hypoglycemic clamp
Blood samples for diabetes status (clamp-induced glucagon, epinephrine, norepinephrine, growth hormone, cortisol and glucose)

Description of Visits—Main Study, Treatment Arm 2

Visit 1, Screening (Day −14 to −28)

Before any study specific procedures may be performed, the patients must sign the informed consent form (ICF). Patients that were included in the Safety and Dose Escalation study will be offered to take part in the main study and will not need to sign a new informed consent or perform a new screening visit but will have a washout period for at least 1 month. Potential study patients will receive both written and oral information about the study procedures, potential risks, and benefits. After written informed consent has been obtained, the following activities and assessments will be performed and any SAEs occurring after signing the ICF will be recorded:

Eligibility will be checked versus the inclusion/exclusion criteria
Demographics (including age and gender)
Medical history
Family history of T1D
General Physical Examination, including weight and height
Neurological assessment
ECG
Concomitant medication
Vital signs (Blood Pressure [BP])
Blood sampling for hematology and clinical chemistry
Urine analysis for microalbuminuria and creatinine
Blood samples for diabetes status (fasting C-peptide, HbA1c and fasting glucose)
Glycemic variability/fluctuations device (FreeStyle LibrePro, FGM) will be given to the patient
The patient will be given a patient diary and instructed to collect their blood glucose and insulin dose for 3 days prior to the next visit

Examinations will be performed according to Table 7

Visit 2, Baseline (Day 1)

At Visit 2 (Baseline, Day 1) the following activities and assessments will be performed:
Eligibility will be checked versus the inclusion/exclusion criteria
Randomization into treatment arm 1, 2 or 3
General Physical Examination, including weight
Neurological assessment
Concomitant medication
Vital signs (BP)
Blood sampling for hematology, clinical chemistry, GABA levels and immunological analyses and PBMC isolation
Urine analysis for microalbuminuria and creatinine
Patient will be asked to provide stool samples
Mixed Meal Tolerance Test (MMTT)
Blood samples for diabetes status (fasting C-peptide, MMTT-induced C-peptide, HbA1c, fasting glucose and MMTT-induced glucose, insulin, proinsulin, glucagon, GLP-1 and lipids)
QoL questionnaire
Blood glucose and insulin dose collected (Exogenous insulin dose/kg/24 hours) for 3 days prior to visit
Self-reported severe hypoglycemia (defined as needing help from others and/or seizures and/or unconsciousness)

Examinations will be performed according to Table 7

Visit 3, Baseline (Day 2)

At Visit 3 (Baseline, Day 2) the following activities and assessments will be performed:
Hypoglycemic clamp
Blood samples for diabetes status (clamp-induced glucagon, epinephrine, norepinephrine, growth hormone, cortisol and glucose)

Examinations will be performed according to Table 7

Visit 4 (Day 44)

At Visit 4 (Day 44) the following activities and assessments will be performed:
General Physical Examination, including weight
Neurological assessment
Concomitant medication
Vital signs (BP)
Blood sampling for hematology, clinical chemistry, GABA levels
Urine analysis for microalbuminuria and creatinine
Patient will be asked to provide stool samples?
Mixed Meal Tolerance Test (MMTT)
Blood samples for diabetes status (fasting C-peptide, MMTT-induced C-peptide, HbA1c, fasting glucose and MMTT-induced glucose, insulin, proinsulin, glucagon, GLP-1 and lipids)
QoL questionnaire
Blood glucose and insulin dose collected (Exogenous insulin dose/kg/24 hours) for 3 days prior to visit
Self-reported severe hypoglycemia (defined as needing help from others and/or seizures and/or unconsciousness)
Treatment with GABA will start. Remind the patient that the daily dose of GABA should be taken with food in the morning, except for days when they come in for a study visit.

Examinations will be performed according to Table 7

Visit 5 (Day 51)

At Visit 5 (Day 51) the following activities and assessments will be performed:
General Physical Examination, including weight
Neurological assessment
Concomitant medication
Vital signs (BP)
AEs
Blood sampling for hematology, clinical chemistry, GABA levels and fasting glucose
Urine analysis for microalbuminuria and creatinine
Blood glucose and insulin dose collected (Exogenous insulin dose/kg/24 hours) for 3 days prior to visit
Self-reported severe hypoglycemia (defined as needing help from others and/or seizures and/or unconsciousness)

Examinations will be performed according to Table 7

Visit 6 (Day 74)

At Visit 6 (Day 74) the following activities and assessments will be performed:
General Physical Examination, including weight
Neurological assessment
ECG
Concomitant medication
Vital signs (BP)
AEs
Blood sampling for hematology, clinical chemistry, GABA levels, and immunological analyses
Urine analysis for microalbuminuria and creatinine
Blood samples for diabetes status (fasting C-peptide, HbA1c, fasting glucose, glucagon, GLP-1, lipids
Blood glucose and insulin dose collected (Exogenous insulin dose/kg/24 hours) for 3 days prior to visit
Self-reported severe hypoglycemia (defined as needing help from others and/or seizures and/or unconsciousness)

Examinations will be performed according to Table 7

After Visit 6 a DSMB will evaluate the safety data from the first 4 patients completing 1 month of treatment with the high dose GABA and evaluate if the treatment can continue.

Visit 7 and 8 (Day 134 and 224)

At Visit 7 (Day 134) and Visit 8 (Day 224) the following activities and assessments will be performed:
General Physical Examination, including weight
Neurological assessment
Concomitant medication
Vital signs (BP)
AEs
Blood sampling for hematology, clinical chemistry, GABA levels, immunological analyses and PBMC isolation (PBMCs will only be collected at visit 8)
Urine analysis for microalbuminuria and creatinine
Patient will be asked to provide stool samples
Mixed Meal Tolerance Test (MMTT)
Blood samples for diabetes status (fasting C-peptide, MMTT-induced C-peptide, HbA1c, fasting glucose and MMTT-induced glucose, insulin, proinsulin, glucagon, GLP-1 and lipids)
QoL questionnaire (Visit 8)
Blood glucose and insulin dose collected (Exogenous insulin dose/kg/24 hours) for 3 days prior to visit
Glycemic variability/fluctuations device (FreeStyle LibrePro, FGM) will be given to the patient (Visit 8)
Self-reported severe hypoglycemia (defined as needing help from others and/or seizures and/or unconsciousness)

Examinations will be performed according to Table 7

Visit 9 (Day 225)

At Visit 9 (Day 225) the following activities and assessments will be performed:
Hypoglycemic clamp
Blood samples for diabetes status (clamp-induced glucagon, epinephrine, norepinephrine, growth hormone, cortisol and glucose)
Treatment with GABA will end.

Examinations will be performed according to Table 7

Visit 10, Follow Up (Day 254)

At Visit 10 (Follow Up, Day 254) the following activities and assessments will be performed:
General Physical Examination, including weight
Neurological assessment
ECG
Concomitant medication
AEs
Blood sampling for hematology, clinical chemistry, GABA levels, immunological analyses and PBMC isolation
Urine analysis for microalbuminuria and creatinine
Patient will be asked to provide stool samples
Mixed Meal Tolerance Test (MMTT)
Blood samples for diabetes status (fasting C-peptide, MMTT-induced C-peptide, HbA1c, fasting glucose and MMTT-induced glucose, insulin, proinsulin, glucagon, GLP-1 and lipids)
QoL questionnaire
Blood glucose and insulin dose collected (Exogenous insulin dose/kg/24 hours) for 3 days prior to visit
Glycemic variability/fluctuations device (Freestyle LibrePro, FGM) will be returned by the patient
Self-reported severe hypoglycemia (defined as needing help from others and/or seizures and/or unconsciousness)

Examinations will be performed according to Table 7

Visit 11, Follow Up (Day 255)

At Visit 11 (Follow Up, Day 255) the following activities and assessments will be performed:
Hypoglycemic clamp
Blood samples for diabetes status (clamp-induced glucagon, epinephrine, norepinephrine, growth hormone, cortisol and glucose)

Examinations will be performed according to Table 7

Description of Visits—Main Study, Treatment Arm 3

Visit 1, Screening (Day −14 to −28)

Before any study specific procedures may be performed, the patients must sign the informed consent form (ICF). Patients that were included in the Safety and Dose Escalation study will be offered to take part in the main study and will not need to sign a new informed consent or perform a new screening visit but will have a washout period for at least 1 month. Potential study patients will receive both written and oral information about the study procedures, potential risks, and benefits. After written informed consent has been obtained, the following activities and assessments will be performed and any SAEs occurring after signing the ICF will be recorded:

Eligibility will be checked versus the inclusion/exclusion criteria
Demographics (including age and gender)
Medical history
Family history of T1D
General Physical Examination, including weight and height
Neurological assessment
ECG
Concomitant medication
Vital signs (Blood Pressure [BP])
Blood sampling for hematology and clinical chemistry
Urine analysis for microalbuminuria and creatinine
Blood samples for diabetes status (fasting C-peptide, HbA1c and fasting glucose)
Glycemic variability/fluctuations device (Freestyle LibrePro, FGM) will be given to the patient
The patient will be given a patient diary and instructed to collect their blood glucose and insulin dose for 3 days prior to the next visit

Examinations will be performed according to Table 8

Visit 2, Baseline (Day 1)

At Visit 2 (Baseline, Day 1) the following activities and assessments will be performed:
Eligibility will be checked versus the inclusion/exclusion criteria
Randomization into treatment arm 1,2 or 3
General Physical Examination, including weight
Neurological assessment
Concomitant medication
Vital signs (BP)
Blood sampling for hematology, clinical chemistry, GABA levels and immunological analyses and PBMC isolation
Urine analysis for microalbuminuria and creatinine
Patient will be asked to provide stool samples
Mixed Meal Tolerance Test (MMTT)
Blood samples for diabetes status (fasting C-peptide, MMTT-induced C-peptide, HbA1c, fasting glucose and MMTT-induced glucose, insulin, proinsulin, glucagon, GLP-1 and lipids)
QoL questionnaire
Blood glucose and insulin dose collected (Exogenous insulin dose/kg/24 hours) for 3 days prior to visit
Self-reported severe hypoglycemia (defined as needing help from others and/or seizures and/or unconsciousness)

Examinations will be performed according to Table 8

Visit 3, Baseline (Day 2)

At Visit 3 (Baseline, Day 2) the following activities and assessments will be performed:
Hypoglycemic clamp
Blood samples for diabetes status (clamp-induced glucagon, epinephrine, norepinephrine, growth hormone, cortisol and glucose)

Examinations will be performed according to Table 8

Visit 4 (Day 44)

At Visit 4 (Day 44) the following activities and assessments will be performed:
General Physical Examination, including weight
Neurological assessment
Concomitant medication
Vital signs (BP)
Blood sampling for hematology, clinical chemistry, GABA levels
Urine analysis for microalbuminuria and creatinine
Patient will be asked to provide stool samples
Mixed Meal Tolerance Test (MMTT)
Blood samples for diabetes status (fasting C-peptide, MMTT-induced C-peptide, HbA1c, fasting glucose and MMTT-induced glucose, insulin, proinsulin, glucagon, GLP-1 and lipids)
QoL questionnaire
Blood glucose and insulin dose collected (Exogenous insulin dose/kg/24 hours) for 3 days prior to visit
Self-reported severe hypoglycemia (defined as needing help from others and/or seizures and/or unconsciousness)
Treatment with GABA and Alprazolam will start. Remind the patient that the daily dose of GABA and Alprazolam should be taken with food in the morning, except for days when they come in for a study visit.
Tablet intake days 44-46 will each be followed by a 3 h observation period at the ward.

Examinations will be performed according to Table 8

Visit 5 (Day 51)

At Visit 5 (Day 51) the following activities and assessments will be performed:
General Physical Examination, including weight
Neurological assessment
Concomitant medication
Vital signs (BP)
AEs
Blood sampling for hematology, clinical chemistry, GABA levels and fasting glucose
Urine analysis for microalbuminuria and creatinine
Blood glucose and insulin dose collected (Exogenous insulin dose/kg/24 hours) for 3 days prior to visit
Self-reported severe hypoglycemia (defined as needing help from others and/or seizures and/or unconsciousness)

Examinations will be performed according to Table 8

Visit 6 (Day 74)

At Visit 6 (Day 74) the following activities and assessments will be performed:
General Physical Examination, including weight
Neurological assessment
ECG
Concomitant medication
Vital signs (BP)
AEs
Blood sampling for hematology, clinical chemistry, GABA levels, and immunological analyses
Urine analysis for microalbuminuria and creatinine
Blood samples for diabetes status (fasting C-peptide, HbA1c, fasting glucose, glucagon, GLP-1, lipids
Blood glucose and insulin dose collected (Exogenous insulin dose/kg/24 hours) for 3 days prior to visit
Self-reported severe hypoglycemia (defined as needing help from others and/or seizures and/or unconsciousness)

Examinations will be performed according to Table 8

After Visit 6 a DSMB will evaluate the safety data from the first 4 patients completing 1 month of treatment with the high dose GABA in combination with Alprazolam and evaluate if the treatment can continue.

Visit 7 (Day 134)

At Visit 7 (Day 134) the following activities and assessments will be performed:
General Physical Examination, including weight
Neurological assessment
Concomitant medication
Vital signs (BP)
AEs
Blood sampling for hematology, clinical chemistry, GABA levels, immunological analyses and PBMC isolation (PBMCs will only be collected at visit 8)
Urine analysis for microalbuminuria and creatinine
Patient will be asked to provide stool samples
Mixed Meal Tolerance Test (MMTT)
Blood samples for diabetes status (fasting C-peptide, MMTT-induced C-peptide, HbA1c, fasting glucose and MMTT-induced glucose, insulin, proinsulin, glucagon, GLP-1 and lipids)
QoL questionnaire (Visit 8)
Blood glucose and insulin dose collected (Exogenous insulin dose/kg/24 hours) for 3 days prior to visit
Glycemic variability/fluctuations device (FreeStyle LibrePro, FGM) will be given to the patient (Visit 8)
Self-reported severe hypoglycemia (defined as needing help from others and/or seizures and/or unconsciousness)
Instruct the patient to stop taking Alprazolam, but to continue with GABA until visit 9.

Examinations will be performed according to Table 8

Visit 8 (Day 224)

At Visit 8 (Day 224) the following activities and assessments will be performed:
General Physical Examination, including weight
Neurological assessment
Concomitant medication
Vital signs (BP)
AEs
Blood sampling for hematology, clinical chemistry, GABA levels, immunological analyses and PBMC isolation (PBMCs will only be collected at visit 8)
Urine analysis for microalbuminuria and creatinine
Patient will be asked to provide stool samples
Mixed Meal Tolerance Test (MMTT)
Blood samples for diabetes status (fasting C-peptide, MMTT-induced C-peptide, HbA1c, fasting glucose and MMTT-induced glucose, insulin, proinsulin, glucagon, GLP-1 and lipids)
QoL questionnaire (Visit 8)
Blood glucose and insulin dose collected (Exogenous insulin dose/kg/24 hours) for 3 days prior to visit
Glycemic variability/fluctuations device (FreeStyle LibrePro, FGM) will be given to the patient (Visit 8)
Self-reported severe hypoglycemia (defined as needing help from others and/or seizures and/or unconsciousness)

Examinations will be performed according to Table 8

Visit 9 (Day 225)

At Visit 9 (Day 225) the following activities and assessments will be performed:
Hypoglycemic clamp
Blood samples for diabetes status (clamp-induced glucagon, epinephrine, norepinephrine, growth hormone, cortisol and glucose)
Treatment with GABA will end.

Examinations will be performed according to Table 8

Visit 10, Follow Up (Day 254)

At Visit 10 (Follow Up, Day 254) the following activities and assessments will be performed:
General Physical Examination, including weight
Neurological assessment
ECG
Concomitant medication
AEs
Blood sampling for hematology, clinical chemistry, GABA levels, immunological analyses and PBMC isolation
Urine analysis for microalbuminuria and creatinine
Patient will be asked to provide stool samples
Mixed Meal Tolerance Test (MMTT)
Blood samples for diabetes status (fasting C-peptide, MMTT-induced C-peptide, HbA1c, fasting glucose and MMTT-induced glucose, insulin, proinsulin, glucagon, GLP-1 and lipids)
QoL questionnaire
Blood glucose and insulin dose collected (Exogenous insulin dose/kg/24 hours) for 3 days prior to visit
Glycemic variability/fluctuations device (FreeStyle LibrePro, FGM) will be returned by the patient
Self-reported severe hypoglycemia (defined as needing help from others and/or seizures and/or unconsciousness)

Examinations will be performed according to Table 8

Visit 11, Follow Up (Day 255)

At Visit 11 (Follow Up, Day 255) the following activities and assessments will be performed:
Hypoglycemic clamp
Blood samples for diabetes status (clamp-induced glucagon, epinephrine, norepinephrine, growth hormone, cortisol and glucose)

Examinations will be performed according to Table 8

Patient Diary

The patients will be supplied with a patient diary starting from the screening visit (Appendix 1). The patient will be carefully instructed by study personnel on how to use the diary and what information to enter. From Visit 2 until the second last visit in the study the patient will record their blood glucose levels (either by finger prick or if the patient has a FGM/CGM device) and insulin doses 3 days before each visit. The patient will also use the diary for documentation of any illnesses, severe hypoglycemias and any concomitant medication that might have been used. During each visit the diary will be reviewed by the Investigator/Study nurse.

Study Stopping Criteria

The Sponsor and the investigators reserve the right to discontinue the study at any time for safety reasons or other reasons jeopardizing the justification of the study. Such a termination will be implemented in a time frame that is compatible with the patient's wellbeing.

If the study is prematurely terminated or suspended, the investigator should promptly inform the patients and assure appropriate therapy and follow-up. The Sponsor will notify the Regulatory Authorities and the IEC of any plans to terminate the study.

Decision Criteria

A patient is included in the study when the ICF has been signed at the screening visit (Visit 1). No patients will be enrolled until approvals from the appropriate Independent Ethics Committee (IEC) and Regulatory Authority have been obtained.

Prior to the study starting, it will be confirmed that all regulatory requirements for starting the study are met. All key documents must be on file in the Sponsor and Investigator's File, respectively.

Management of the Disease

All patients will continue to receive intensive insulin treatment through their personal physicians via multiple daily injections of insulin or via insulin pump and should not use any oral or injected non-insulin pharmaceuticals for glycemic control. If, by mistake, such medication is introduced during the study, this is not a reason for discontinuation of the patient. The primary responsibility for diabetes management will be the treating or referring diabetes care provider, but the research study team will provide close additional support through interaction by phone as needed. Diabetes management will be monitored by the study staff with phone calls between study visits as needed. Patients will record their insulin doses for 3 days before each visit.

The following diabetes treatment goals will be given to patients and treating physicians in accordance with the current recommendations of the American Diabetes Association (ADA)
Pre-meal glucose 4.4 to 7.2 mmol/L
Bedtime/overnight glucose 5 to 8.3 mmol/L
HbA1c<53 mmol/mol (Diabetes Control and Complications Trial [DCCT]-aligned)

These goals are only for guidance and are not considered as violating the protocol if not achieved, but it is regarded as important that every patient and treating physician make their best effort to achieve these recommendations.

Data Safety Monitoring Board

An independent DSMB will be appointed. The DSMB will evaluate the first patient upon completion of all the dose escalation steps and decide if the other 5 patients can enter the study. When all 6 patients have completed the Safety and Dose Escalation study the DSMB will again review the safety data and decide if the main study can start.

The DSMB will then evaluate the safety data from the first 4 patients that have completed 1 month of treatment with GABA (Treatment arm 1) and evaluate if Treatment arms 2 and 3 can start or if the study should be terminated due to safety concerns for the patients. The DSMB will then also evaluate the safety data for the first 4 patients that have completed 1 month of treatment in Treatment arms 2 and 3, to make sure that no safety concerns have arisen upon treatment with the higher dose of GABA and the combination of GABA and Alprazolam. A DSMB charter will be written to outline the working procedures and the duties of the DSMB.

Study Population

The study will take place at 1 site in Uppsala, Sweden and will include approximately 24 patients. T1D patients, diagnosed 5 years at the time of screening are given information about the study and are asked to participate in the trial.

Inclusion Criteria
1. Informed consent given by patients according to national regulations
2. Type 1 diabetes diagnosed 5 years at the time of screening
3. Must have been diagnosed with Type 1-diabetes before the age of 25
4. Age and 50
5. Fasting C-peptide levels should be in the range from not detectable levels up to <0.12 nmol/L
6. For males of childbearing potential adequate contraception is as follows:
   a. condom (male)
   b. abstinence from heterosexual intercourse
   c. female partner using contraception as below listed:
oral (except low-dose gestagen (lynestrenol and norestisteron)), injectable, or implanted hormonal contraceptives
combined (estrogen and progestogen containing)
oral, intravaginal or transdermal progesterone hormonal contraception associated with inhibition of ovulation
intrauterine device
intrauterine hormone-releasing system (for example, progestin-releasing coil)
bilateral tubal occlusion

Exclusion Criteria
1. Females of child-bearing potential
2. Previous or current treatment with immunosuppressant therapy (although topical and inhalation steroids are accepted)
3. Treatment with any oral or injected anti-diabetic medications other than insulin
4. Patients on medications which may disturb GABA action, such as Baclofen, Valium, Acamprosate, Neurontin, or Lyrica
5. HbA1c>90 mmol/mol
6. eGFR<60 ml/min
7. Increased plasma concentrations of alanine aminotransferase (>0.75 μkatl/I for females or >1.1 μkat/I for males) and/or aspartate aminotransferase (>0.60 μkat/I for females or >0.75 μkat/Ifor males).
8. Known cancer disease
9. Known sleeping apnea or pulmonary disorder with carbon dioxide retention in blood
10. Previous history of pancreatitis or other exocrine pancreatic disorder
11. A history of epilepsy, myasthenia gravis, head trauma or cerebrovascular accident, or clinical features of continuous motor unit activity in proximal muscles
12. A history of alcohol or drug abuse
13. A significant illness other than diabetes within 2 weeks prior to first dosing
14. Known human immunodeficiency virus (HIV) or hepatitis
15. Females who are breastfeeding
16. Males not willing to use adequate contraception during the study period.
17. Known hypersensitivity against benzodiazepins or any excipients of study drugs
18. Participation in other clinical trials with a new chemical entity within 3 months or 5 half-lives of the new chemical entity, whatever longest.
19. Inability or unwillingness to comply with the provisions of this protocol
20. Deemed by the investigator not being able to follow instructions and/or follow the study protocol or other reasons that, at the investigator's discretion, could affect the subject's current clinical condition during study procedures.

Recruitment and Screening

Patients between the ages and 50 who received their Type 1-diabetes diagnosis 5 years ago will be asked to participate in the study. Eligible patients will have the study explained to them, and will receive the written patient information. After having had the time to review the nature of the study, they will have the opportunity to ask questions to the investigational team. If, after this, the patients agree to participate, they will personally sign and date the written ICF. Patients will provide written informed consent before any study-related procedures are performed.

The patients will then receive a copy of the signed and dated patient information/ICF.

Re-Screening of Patients

Under certain circumstances patients are allowed to be rescreened once at the investigators discretion. Examples of such circumstance could be, but not exclusively, the patients HbA1c decreases below 90 mmol/mol, a male that plans to have children and the partner becomes pregnant.

Patient Withdrawal Criteria

The patient will receive oral and written information about the study, which includes information about the right to withdraw from the trial at any time without prejudice to future treatment. In addition, the patient may be withdrawn at the investigator's or Sponsor's discretion at any time if regarded in the patient's best interest. In the event that the patient drops out of the trial or is withdrawn from the trial, the appropriate withdrawal page (Study Termination Report) in the CRF must be completed. Please note that a Study Termination Report must be completed for all patients who have given informed consent and who have been assigned a patient number.

Reasonable efforts should be made to contact any patient lost to follow-up during the course of the trial in order to complete assessments and retrieve any outstanding data.

Patients withdrawn after randomization will not be replaced.

Note that any changes in insulin requirement are not a reason for withdrawal of the patient from the study.

Patient Withdrawal

In accordance with the Declaration of Helsinki, the investigator must explain to the patient that they have the right to withdraw from the study at any time, and that this will in no way prejudice their future treatment. The reason for any kind of withdrawal must be recorded on the appropriate section of the CRF.

There will be two main categories for withdrawals from the study:

Complete withdrawal (i.e. stopping investigational product(s) and also continued efficacy and safety evaluations). In order to achieve as complete as possible 9-12-month follow-up (depending on treatment arm), whenever feasible, patients that are considered for complete withdrawal should be asked if they could consider to return for a 9 or 12-month visit (depending on treatment arm), preferable complete visit but as a minimum do the MMTT, C-peptide and HbA1c sampling, and collection of AEs. Attempts should be made to contact patients lost to follow-up with respect to this.

Standard reasons for withdrawing from further participation in the study and from the follow-up visits may be:
Patient's decision (withdrawal of consent to participate)
Patient lost to follow-up

Withdrawals from investigational product(s) (i.e. stopping one or several investigational products, but continuing follow-up visits, including efficacy and safety evaluations)

Standard reasons from withdrawing from taking further investigational product, but continuing follow-up visits and safety evaluations may be:
Unacceptable AEs
Patient request
Investigator's discretion
Patient lost to follow-up/non-attendance
Intercurrent illness

GABA should not be given to the patient if the patient after inclusion in the study develops/experiences/receives:
Brain damage, epilepsy, head trauma, neurological disease
Immune-suppressive treatment
Cancer, cancer treatment
Pancreatitis
Any other diabetes drugs other than insulin (and the combination regimen administered in this study)
Treatments with medication that disturbs GABA action, such as Baclofen, Valium, Acamprosate, Neurontin, or Lyrica
Drug/alcohol abuse

Alprazolam should not be given to the patient if the patient after inclusion in the study develops/experiences/receives:
Drug/alcohol abuse
Any analgesic that contains opioid analogues
Sleep apnea or pulmonary disorder with carbon dioxide retention in blood
Myasthenia gravis
Any symptoms of overdose i.e.; dizziness, drowsiness, difficulty breathing, confusion, muscle weakness, unconsciousness, impaired coordination, agitation, aggressiveness, hallucinations etc.

However, whenever a patient is withdrawn from a study, or for whatever reason is not coming to any further visits, a final study evaluation must be completed for that patient stating the reason(s) why the patient was withdrawn from the study. All documentation concerning the patient must be as complete as possible.

Withdrawals due to non-attendance must be followed up by the investigator to obtain the reason for non-attendance. Withdrawals due to intercurrent illnesses or AEs must be fully documented in the CRF, with the addition of supplementary information if available and/or appropriate.

Study Assessments

Assessment of Safety Variables

The safety assessments include:
Occurrence of AEs
Laboratory measurements (hematology and clinical chemistry)
Urine analysis
Physical examinations, including neurological assessments
Vital signs

Laboratory Safety Assessments
Hematology: (mean corpuscular hemoglobin [MCH], mean corpuscular volume [MCV], mean corpuscular hemoglobin concentration [MCHC], Hemoglobin, Platelets, Total Leukocyte Count and Differential)
Clinical Chemistry: Creatinine, Calcium, Liver function tests (alanine aminotransferase [ALT], Aspartate Aminotransferase [AST], Alkaline Phosphatase [ALP], Bilirubin)
Urine analysis: Microalbuminuria Creatinine

The outcome of the assessments will be recorded as “normal” or “abnormal”. Abnormal findings (values outside the reference ranges) will be assessed as “clinically significant” or “not clinically significant”.

The timing of the assessments is described in Above.

The laboratory tests listed above (hematology, clinical chemistry and urine analysis) will be analyzed at Uppsala University Hospital. Full details on blood sample collection, processing and storage, are provided in the study specific Laboratory Manual.

Adverse Events

AEs will be recorded throughout the study period from first dose of study drugs (Visit 3, Treatment arm 1 and Visit 4, Treatment arms 2 and 3) to last study visit. Events occurring until the day before first oral intake of investigational medicinal product (IMP) will be recorded as medical history.

SAEs will be recorded from signing the ICF.

AEs will be coded using the current version of MedDRA.

Physical Examination

All patients will undergo a general physical examination (general appearance including skin, mouth, throat, cardiovascular, abdomen, lymphatic glands, and neurological/musculoskeletal [including reflexes]).

The timing of the assessments is described above.

The outcome of the assessments will be recorded as “normal” or “abnormal”. If the outcome is assessed as abnormal, the finding will be classified as “clinically significant” or “non-clinically significant”.

Any findings until first dose of GABA (Visit 3, Treatment arm 1 and Visit 4, Treatment arms 2 and 3) will be reported as pre-existing conditions on the Medical History CRF page.

During the subsequent study visits the patient will be examined for any new medical conditions or worsening of the pre-existing ones. Any change in pre-existing conditions or new conditions must be entered on the AE page in the CRF and any medication given on the concomitant medication pages.

Neurological Examination

All patients will undergo a standardized clinical neurological examination at some visits. The timing of the assessments is described above.

The neurological tests are performed in order to detect possible mild signs of neuromuscular disease such as disturbance of strength, balance, and coordination.

The outcome of the assessments will be recorded as “normal” or “abnormal”. Abnormal findings will be assessed as “clinically significant” or “not clinically significant”.

The neurological examination includes:
Extremity reflexes
Romberg (balance and coordination)
Walk on a line, 2 meters (balance and coordination)
Standing on 1 leg, left and right, 15 seconds per leg (balance and coordination)
Finger-nose (coordination)
Mimic (cranial nerves)
Babinski reflex (central function)
Muscle strength (shake hands) biceps, triceps, distal extensors, and flexors

These examinations may also be repeated between scheduled visits at the discretion of the investigator. Screening for neurological disease with electroencephalogram (EEG) is not included due to low sensitivity and specificity. However, if any signs of neurological dysfunction are detected, the patient should be referred to a neurologist for further evaluation.

During the study visits the patient will be examined for any new medical conditions or worsening of the pre-existing ones. Any change in pre-existing conditions or new conditions must be entered on the AE page in the CRF.

Electrocardiography

Electrocardiography (ECG) will be recorded as a part of the general physical examination.

The timing of the assessment is described in above.

Vital Signs The following vital signs will be monitored as safety variables:
Supine systolic and diastolic blood pressure (mmHg), after 5 minutes lying down Weight, height and BMI will also be recorded.

The outcome of the assessments will be recorded as “normal” or “abnormal”. Abnormal findings will be assessed as “clinically significant” or “not clinically significant”. The timing of the assessments is described in Above.

Assessment of Variables of Diabetes Status

Mixed Meal Tolerance Test

Meal stimulated glucose and C-peptide will be assessed using the MMTT. In the Main study, insulin and proinsulin concentrations will also be measured. The MMTT must be performed according to the instructions in the Operations Manual (Appendix 2).

The timing of the assessments is described in Above.

The patient should:
Come to the study site following an overnight fast (>10 hours), i.e. the patient may not eat but is permitted to drink water
Not have taken the Remygen and, if applicable, Alprazolam dose in the morning
Not take short acting/direct acting insulin within 6 hours before the MMTT. The patient is allowed to take base-insulin day/night before, but not in the morning before the MMTT.
Patients with CSII (insulin pump) must continue with their basal dose insulin, but not add bolus dose during the last 6 hours before the MMTT
Have a fasting plasma glucose level in the range defined by 4-12 mmol/L on the patient's home blood glucose meter in the morning of the test

If the patient does not fulfill all of the above criteria, the MMTT should be rescheduled and the patient should return to the study site within 5 days if possible.

If a patient has a blood sugar level <4 mmol/L in the morning of the scheduled visit, the patient is allowed to take dextrose tablets and then come to the study site. The patient is allowed to perform the MMTT if the blood sugar level is >4 mmol/L when the patients arrive at the study site. If the blood sugar level is still <4 mmol/L the visit should be rescheduled according to the instructions below.

For patients with clinical signs of ketoacidosis or unequivocal elevation of fasting plasma glucose (>12 mmol/L [216 mg/dl]) on the patient's home blood glucose meter, the complete visit should be postponed and the patient should return to the study site within 5 days of the scheduled visit. Please note that all study procedures/examinations must be postponed.

If for safety reasons, patients need to eat or take insulin, the visit should also be rescheduled.

Patients are fasting for 10 hours before test, which is then performed in the morning thereafter. In the dose-escalation part of the study, the study drug will be taken 30 min prior to the mixed meal tolerance test, whereas in the main study the study drug will be taken after finalization of the test. Before start, p-glucose should be in the range 4-12 mmol/I.

A venous catheter is inserted for blood retrieval during test. Analyses of blood for p-glucose and C-peptide (insulin and proinsulin) is then performed before (time 0) and 15, 30, 60, 90 and 120 min after oral intake of 360 ml of Resource protein, Nestle′ for min.

Hypoglycemic Clamp

In order to evaluate the counterregulatory hormone response to hypoglycemia, a hypoglycemic clamp will be performed. The Hypoglycemic Clamp must be performed according to the instructions in the Operations Manual. (Appendix 3)

The timing of the assessments is described in Above.

The patient should:
Come to the study site following an overnight fast (>10 hours), i.e. the patient may not eat but is permitted to drink water
Not have taken the Remygen and, if applicable, Alprazolam dose in the morning
Not take short acting/direct acting insulin within 6 hours before the investigation.
The patient is allowed to take base-insulin day/night before, but not in the morning before the investigation.

Patients with CSII (insulin pump) must continue with their basal dose insulin, but not add bolus dose during the last 6 hours before the investigation.

If a patient has a blood sugar level <4 mmol/L in the morning of the scheduled visit, the patient is allowed to take dextrose tablets and then come to the study site. The patient is allowed to perform the investigation if the blood sugar level is >4 mmol/L.

In the dose-escalation part of the study, the patient will take study drug 30 min prior to start of the hypoglycemic clamp. In the main study, the study drug will instead be taken after finalization of the hypoglycemic clamp. Baseline blood samples will be obtained for glucose, glucagon, epinephrine, norepinephrine, growth hormone and cortisol. The clamp will then be initiated by an intravenous insulin infusion at a rate of 2 mU×kg⁻¹×min⁻¹. Glucose 100 mg/ml will then be administered in variable infusion rates to clamp the blood glucose concentration at 5.5 mmol/land then at 2.5 mmol/land kept stable for 30 min. Blood glucose will be evaluated repeatedly in order to find the optimal glucose infusion rate. Blood sampling to determined levels of counterregulatory hormones as described above will be performed at each of these target levels when a stable glucose concentration has been reached. The insulin infusion will thereafter be terminated and the patient is allowed to eat and will be observed until normoglycemia is achieved. If necessary, additional glucose administration will be given to allow blood glucose concentrations to normalize.

Hemoglobin A1c

Blood samples will be taken to measure HbA1c levels and will be analyzed at Uppsala University Hospital. The timing of the assessments is described in Above.

Fasting Glucose and Fasting C-Peptide

Blood samples will be taken to measure fasting glucose and fasting C-peptide levels and will be analyzed at Uppsala University. The timing of the assessments is described in Above.

Glucagon, Glucagon Like Peptide-1, Lipids

Blood samples will be taken to measure plasma levels of glucagon, glucagon like peptide-1 (GLP-1) and lipids and will be analyzed at Uppsala University Hospital. The timing of the assessments is described in Section 7.2-7.5 and Table 55-8.

Glycemic Fluctuations/Variability

The FreeStyle Libre Pro FGM System is a professional glucose monitoring device indicated for detecting trends and tracking patterns and glucose level excursions above or below the desired range, facilitating therapy adjustments in persons with diabetes. Readings from the FreeStyle Libre Pro FGM System are not made available directly to patients in real time. The FreeStyle Libre Pro FGM System aids in the detection of episodes of hyperglycemia and hypoglycemia, facilitating therapy adjustments.

Patients will receive the Freestyle Libre Pro at the site at Visit 1 and Visit 2 in the Safety and Dose Escalation study and will be instructed how to return the FreeStyle Libre to the site after each of the testing period. For the main study patients will receive the Freestyle Libre Pro at the site during Visit 1 (all Treatment arms), Visit 7 (Treatment arm 1), Visit 8 (Treatment arm 2 and 3) and will be instructed how to return the FreeStyle Libre to the site after each of the testing period. However, for Visit 9 (Treatment arm 1) and Visit 10 (Treatment arm 2 and 3) patients will be instructed to come to the site 14 days before the visit to receive the Freestyle Libre and have it activated. The patients will then return the Freestyle Libre at Visit 9 or Visit 10, depending on treatment arm. The timing of the distribution of the glucose monitoring system and assessments is described in Above.

Immune System Assessments

Bioanalytical Method

Assessment of Serum Autoantibodies, Cytokine Analysis and Profiling of the Immune Cells

Peripheral blood will be taken from the patient. GAD and IA2 antibodies will be analysed at the central clinical chemistry laboratory at Uppsala University Hospital. A rise in autoantibodies could potentially indicate that GABA treatment has induced regeneration of some β-cells and as a consequence the autoimmune response is again triggered.

In addition, circulating levels of different cytokines in plasma will be analysed and peripheral blood mononuclear cells (PBMCs) will be isolated and characterized.

Cytokine profile is assessed by measuring circulating levels of different cytokines (including IL-2, IL-6, IL-10, IL-17, IL-21, IL-33, IL-35, IFN-gamma and TGF-beta).

PBMC profile is assessed by determining the proportion of different immune cells such as; antigen presenting cells, T-cells, regulatory T cells, regulatory B cells and other innate immune cells by using flow cytometry. Also, PBMCs will be used for determining the immune regulatory genes expression and in vitro assays.

Separate blood plasma will be obtained in EDTA-tubes by centrifugation and immediately frozen. Blood samples for immunology test will be separated to plasma and peripheral blood mononuclear cells (PBMCs). Plasma samples will be stored at −80° C. Isolated PBMCs will be cryopreserved.

Circulating levels of different cytokines (including IL-2, IL-6, IL-10, IL-17, IL-21, IL-33, IL-35, IFN-gamma and TGF-beta) will be analysed in plasma samples by using commercially available ELISA and Luminex techniques (RnD Systems, Abingdon, UK; Cloud-Clone Corp., Houston, USA) at Uppsala University. The PBMCs will be further studied for determining the proportion of different immune cells such as; antigen presenting cells, T-cells, regulatory T cells, regulatory B cells and other innate immune cells by using flow cytometry. Also, PBMCs will be used for determining the immune regulatory genes expression and in vitro assays at Uppsala University.

GABA Levels in Plasma

Blood samples will be taken to measure plasma levels of GABA upon treatment. Samples will be analyzed at Uppsala University. During the Safety and Dose Escalation study the pharmacokinetic profile of GABA in plasma will be evaluated. To do this blood samples will be taken at 0, 30, 60, 90, 120, 180, 300 minutes and 24 h, after 1 dose of GABA at the indicated dose. In the main study, GABA in plasma will be measured through trough concentration sampling. The timing of the assessments is described in Above.

Stool Samples

The patients will be asked to provide stool samples. These samples will be used to analyze how much of the GABA that is passed through the intestines unmetabolized. The timing of the assessments is described in Section 7.3-7.5 and Table 6-8.

Urine Samples

Urine samples will also be collected during the Safety and Dose Escalation study to analyze how much GABA that is excreted through the urine. The timings of the assessment are described in section 7.2 and Table 5.

Assessment of Quality of Life

Quality of Life will be assessed with the Diabetes Treatment Satisfaction Questionnaire (DTSQ) (Appendix 4) and the RAND-36 questionnaire (Appendix 5). The timings of the assessments are described in 7.2-7.5 and Table 5-8.

Assessment of Concomitant Medication

Any concomitant medication must be recorded in the CRF. Note: if a new medication is introduced or an existing medication is changed due to a new medical condition or worsening of a pre-existing medical condition, the condition must be reported as an AE.

The timings of the assessments are described above.

Demographics and Other Baseline Characteristics

Demographics and Baseline Data

The following demographic and baseline data will be collected at the Screening visit (Visit 1):
Age
Sex

Medical History

Medical history will be recorded at the Screening visit (Visit 1).

A complete review of the patient's past medical history will be undertaken by and documented on the Medical History CRF at the screening visit (Visit 1).

All pre-existing conditions/diseases will be reported on the Medical History CRF page at the screening visit (Visit 1). All AEs that occur between Visit 1 and treatment start (Visit 3, arm 1 and Visit 4, arms 2 and 3) will also be considered Medical History.

Medical histories will be coded using the current version of Medical Dictionary for Regulatory Activities (MedDRA).

Family History of T1D

The patient's T1D diagnosis date and family history of T1D will also be documented.

Blood Sampling Procedures

Blood samples for assessments of diabetes status, laboratory safety assessments, GABA levels and immunological testing should be collected in accordance with Table 5-8.

The total volume of blood that will be collected from each patient will be approximately 450-500 mL in the Safety and Dose Escalation study and approximately 600 mL in the Main Study Period.

Treatment of Patients

Study Treatment

Study medication: Remygen oral intake, taken together with food.

Method of Assigning Patients to Treatment Groups

For the main study a randomization list will be produced by a statistician. After confirmation of eligibility at Visit 2 patients will be randomized in a 1:1:1 ratio (Arm 1: Arm 2: Arm 3). Randomization will be stratified by C-peptide level at visit 1 to ensure that an equal number of patients in respective age group are included in each arm.
Arm 1: Low dose of GABA
Arm 2: High dose of GABA
Arm 3: High dose of GABA+Alprazolam

Blinding and Code Breaking

The study is an open label trial, so no blinding and code breaking procedures are needed.

Study Medication

The following medication supplies will be used in the study:

A.

Study medication: Remygen oral intake

Dosage and interval: Dose according to treatment arm, once daily, taken together with food.

IMP supplier: Diamyd Medical AB, Stockholm, Sweden.

B.

Study medication Alprazolam Orion®, oral intake

Dosage and interval: 0.5 mg, once daily, taken together with food.

IMP supplier: Orion Pharma AB, Box 85, 182 11 Danderyd, Sverige

Supply, Packaging, Handling and Storage

Remygen is a disc shaped 500 mg compressed tablet of 10 mm approximate diameter for oral ingestion containing 200 mg active GABA and excipients. Remygen is supplied in sealed bottles containing 40 tablets and a desiccant device. Bottles will be dispensed at appropriate times and in appropriate amounts to the patients by the investigator/study nurse.

Alprazolam Orion is a white oval shaped tablet (size: 9×6 mm) for oral ingestion, containing 0.5 mg of Alprazolam. Alprazolam Orion is supplied in bottles containing 20 tablets. The tablets will be dispensed by the investigator/study nurse every 2 weeks to the patient.

Appropriately labeled bottles of Remygen will be supplied directly to the local pharmacy (Apoteket, Uppsala) for further distribution to the clinic and then to the included patients. Instructions for dosing at home will be given to the patient by the investigator.

Alprazolam will be bought and appropriately labeled by Apoteket, Uppsala and then further distributed to the clinic and the included patients. Instructions for dosing at home will be given to the patient by the investigator.

At the clinic, Remygen and Alprazolam must be stored in a controlled environment at room temperature, 15-25° C., in a secure area (e.g. a locked cabinet or drug storage room), protected from unintended use. Patient must be instructed to keep medication for home use in room temperature and in a secure area (away from children) by the investigator.

All study medication will be labelled with information according to national and local regulations.

Study Medication Accountability

All study medications supplied for this study must be retained in a safe place at all times of the study. Only personnel authorized by the investigator should dispense the study medication to the patient, and the accountability is the responsibility of investigator. The patient should be instructed to return all used (empty medication containers) and unused GABA at Visit 11 (Safety and Dose Escalation study), Visit 8 (treatment arm 1) or Visit 9 (treatment arm 2 and 3).

A study medication inventory (dispensing records) for all medication dispensed must be maintained at all times and always kept current. Used and unused medication must be stored at the site or pharmacy throughout the study. The investigator/pharmacist must keep record of all drugs received, used and returned. The pharmacy and study sites are obliged to properly measure and record the storage temperature.

When the study is completed all unused and used (empty medication containers) GABA study medication containers must be returned to Diamyd Medical unless the drug supplier has approved other arrangements. Unused Alprazolam will be returned to the local pharmacy (Apoteket, Uppsala).

Concomitant Therapy

The patients will receive adequate therapy for concomitant diseases at the discretion of the investigator. Patients receiving a drug listed under exclusion criteria at screening should not be included in the study. Patients who are prescribed a drug listed in the exclusion criteria during the period between screening and start of GABA treatment should be withdrawn from the study. Caution shall be taken to include patients prescribed concomitant medications that have a small therapeutic window, and such medications shall not without caution be introduced in the treatment of the patients.

Any concomitant medication must be recorded in the CRF. Note: if a new medication is introduced or an existing medication is changed due to a new medical condition or worsening of a pre-existing medical condition, the condition must be reported as an AE.

Post Study Treatment

No study medication will be offered to the patients after completion of the final dose. Patients will thereafter receive therapy at the discretion of the investigator and according to prescriptions.

Adverse Events

Definitions of Adverse Events (AE)

An AE is defined as any untoward medical occurrence in a subject during a clinical study administered a medicinal product and which does not necessarily have a causal relationship with this treatment(s).

An AE can therefore be any unfavorable and unintended clinical sign or symptom, any illness or disease, which develops or worsens in intensity during the course of the trial. It also includes an abnormal laboratory finding, if e.g., the abnormality results in trial withdrawal, is serious, is associated with clinical signs or symptoms, or is considered being of clinical relevance.

It could also include accidents and reasons for changes in medication (drug and/or dose), any medical/nursing/pharmacy consultation and admission to hospital/surgical operations.

Any new findings, clinically significant laboratory values or worsening of pre-existing condition must be reported as an AE by the investigator, whether or not considered related to the medicinal product(s).

Note that hospital admission and/or surgical operations for illness, which existed before the study drug was given or the subject was enrolled in the clinical trial and did not worsen during the study, are not AEs.

T1D-Related Events

The number of self-reported episodes of severe hypoglycemia (Severe hypoglycemia defined as needing help from others and/or seizures and/or unconscious) between baseline and subsequent visits will be collected in the CRF. Hypoglycemia, changes in C-peptide, changes to insulin dosage, and increases in HbA1c/blood glucose will not be reported as AEs unless the definition of SAEs is met.

Seriousness

A SAE is defined as: an AE that is fatal, life-threatening, significantly or persistently disabling, requiring hospitalization or prolongation of existing hospitalization or that is a congenital anomaly or birth defect.

These characteristics/consequences have to be considered at the time of the event. For example, regarding a life-threatening event, this refers to an event in which the subject was at risk of death at the time of the event; it does not refer to an event which hypothetically might have caused death if it were more severe.

Some medical events may jeopardize the subject or may require an intervention to prevent one of the above characteristics/consequences. Such events (referred to as important medical events) should also be considered as serious in accordance with the definition.

Intensity

Mild: The AE is transient and easily tolerated.

Moderate: The AE causes the patient discomfort and interrupts the patient's usual activities.

Severe: The AE causes considerable interference with the patient's usual activities, and may be incapacitating or life-threatening.

Note: a distinction should be drawn between serious and severe AEs. The term severe is used to describe the intensity of the event and the event does not necessarily need to be considered serious. The term serious is based on the patient/event outcome or action and serves as a guide for defining regulatory reporting obligations.

Relationship to Study Medication

Relationship to study medication will be assessed. AEs with a causal relationship assessment of Unlikely related, Possibly related and Probably related will be considered to be Adverse Reactions.

Not related: This category is applicable to those AEs which, after careful medical consideration at the time they are evaluated, are judged to be clearly and incontrovertibly due to extraneous causes (disease, environment, etc.) and do not meet the criteria for study medication relationship listed under remote, plausible or probable.

Unlikely related: Time relationship non-existent or doubtful and/or other factor(s) certain or probable to have been causative.

Possibly related: Time relationship exists. Other possible causative factor(s) may exist (e.g., concurrent disease or concomitant medication). Improvement on dechallenges or dose reduction may or may not have been seen.

Probably related: Time relationship exists. No other possible causative factor(s) may exist (not reasonably explained by the patient's known clinical state or concomitant medication). Improvement on dechallenges or dose reduction (if performed) has occurred. Recurrence of symptoms on rechallenge (if performed) has occurred. A specific laboratory investigation (if performed) has confirmed the relationship

Reporting of Adverse Events

All AEs must be recorded in the CRF, defining relationship to study medication, intensity, seriousness, action taken with study drug, and outcome. AEs should also be recorded by the investigator in the patient file/notes.

Timelines and Reporting of SAE

All SAEs must be reported, whether or not considered attributable to the study drug on a separate SAE Report Form. SAEs will be reported from signing of informed consent.

An assigned CRO will be responsible for reporting all SAEs in accordance with ICH Good Clinical Practice (GCP) and local regulations. The sponsor and the CRO will complete and sign a “Working Agreement” agreement covering the safety reporting responsibilities in the study. This agreement will ensure the sponsor is directly informed of each SAE reported by the investigators.

In order to meet the specified reporting requirements investigators should adhere to the following process for recording and reporting SAEs.

It is the investigator's responsibility to, as soon as he/she is aware of a potential SAE, he/she should contact the CRO by fax or e-mail, and in any case no later than 24 hours after the knowledge of such a case.

At the time of initial reporting, the investigator must provide as a minimum requirement, the patient number, birth date, nature of the SAE, and a preliminary assessment of causality. A SAE Report Form should be used for the reporting. If information is missing at the time for the initial reporting a follow-up with additional information on the SAE form should be sent by faxing or e-mailing as soon as possible, preferably no later than within 5 days after the initial report. Numbers/e-mail address are provided in the Investigator Site File and on the SAE Report Form.

Where appropriate, hospitalization or autopsy reports should be made available. All SAEs will be followed up until resolution (i.e., asymptomatic, stabilization or death).

It is the CRO's responsibility to receive e-mail or fax copies of the SAE Report Form and other relevant CRF pages from the investigators. The CRO will review the information provided on the form and enter it into the SAE data base. The SAE report will be assigned a unique number that will be entered on the SAE Report Form, and will be used to identify the report in all future communication. A notification of receipt of the report will be sent to the reporter, either by fax or e-mail within 48 hours. The CRO will contact the investigator directly if there are any inconsistencies or missing information.

The Sponsor is responsible for assessing whether the SAE is classified as a SUSAR. If classified as a SUSAR, the CRO is responsible for the timely submission to the Competent Authority and the IEC according to appropriate requirements. If approved by the Competent Authority the reporting will be done on CIOMS forms, which will be submitted to them by e-mail. The Competent Authority is then responsible for transferring the information electronically to the EudraVigilance database.

Fatal and life-threatening SUSARs should be reported by the CRO as soon as possible to the Competent Authorities and Ethical Committees, and in any case no later than seven (7) calendar days, after knowledge by the Sponsor/CRO of such a case. Relevant follow-up information on the case will be subsequently communicated within an additional eight (8) days. All other SUSARs shall be reported to the Competent Authorities concerned and to the Ethics Committee concerned as soon as possible but within a maximum of fifteen (15) days of first knowledge by the CRO.

Unresolved Events

If an AE/SAE is present when the patient has completed the study the course of the event must be followed until final outcome is known or the condition is stable.

Pregnancy Report Form

Women of child-bearing potential will not be included in the study.

Statistical Methods

Estimation of Sample Size

The main secondary endpoint that will be used to assess efficacy potential will be the proportion of C-peptide values from a 2 hour MMTT that show improvement from baseline after treatment. Our approach maximizes the number of data points available for analysis and is based on the observation that each 120-minute MMTT yields 6 measurements of C-peptide (at times 0, 15, 30, 60, 90, 120 minutes). In addition, each such C-peptide measurement can be classified into one of 3 categories as: “Below 0.05 nmol/I (undetectable)”, “Between 0.05 nmol/I and 0.2 nmol/I”, and “Above 0.2 nmol/I”. The value of 0.2 has been established by the DCCT to be the minimum biologically active level of C-peptide.

Thus, each C-peptide result can be categorized and the change across time can then be summarized in a cross-classification table of “transitions”(Table 9):

TABLE 1
Cross-classification table of transitions

| | After 6 months | | | |
|---|---|---|---|---|
| | | Below 0.05 | Between 0.05 | |
| | | nmol/l | nmol/l and 0.2 | Above 0.2 |
| | | (undetectable) | nmol/l | nmol/l |
| baseline | Below 0.05 | NC | BENEFIT | BENEFIT |
| | (undetectable) | | | |
| | Between 0.05 | HARMFUL | NC | BENEFIT |
| | and 0.2 | | | |

With 6 such classifications per subject and 10 subjects per treatment group, each such treatment-specific table would be based on n=60 observations for a total of 180 observations in this analysis. FIG. 1 below shows the probability our study will successfully detect treatment benefit for a series of hypothetical levels of true benefit and considering different sample sizes for the entire study. That is, each of the sample sizes below represent the numbers of subjects enrolled in both treatment arms of the study. This analysis assumes independence and identical risk probability of the occurrence of a benefit within and across individuals. We suspect there is likely a positive dependence (so that success in one observation for an individual increases the probability of benefit for other observations from that individual) in which case our estimates are conservative. It is also likely that there is individual variation in success probabilities. However, our use of mixed generalized linear modeling will account for that possibility and also yield an estimate of inter-individual differences in likelihood of therapeutic response.

The detection probability is the probability is the probability at least one individual in our study will experience a beneficial improvement in at least one of the 6 C-peptide measurements from the 2-hour MMTT. This has been computed as:

1−BIN(0;180,π)

where “BIN” refers to the Binomial probability density function, with 180 trials and success probability “π”.

We observe that our study has high (80% or greater) likelihood of detecting treatment benefit when the actual rate of benefit is as small as approximately 1.5%. Hence, we conclude that our study is sufficiently powered to successful detect a treatment benefit even when that benefit might occur with fairly small probability.

Statistical Analyses

A detailed statistical analysis plan (SAP) will be written and finalized well in advance of database lock. The plan will follow the outline of the statistical analyses presented below, but details necessary to complete the statistical analyses will be given. We will estimate the true benefit rate in the mixed generalized regression analyses. As a conservative evaluation of statistical precision based on n=10 subjects per treatment (i.e. 60 observations of C-peptide), if the treatment has a 50% beneficial effect, the resulting 95% confidence interval would have +/−13% precision for estimating the true beneficial rate. Since 50% effect is the most conservative choice when determining precision in estimating a proportion, we conclude this sample size provides a very good level of precision for the purposes of detecting and comparing treatment benefit in this early phase treatment protocol. Of course, our sample size evaluation assumes independence between data coming from the same individual which is unlikely. However, as described below, we will use statistical modeling to extract this component of variation when making statistical inference on the proportion of benefit and therefore expect the precision and power to be greatly improved.

We will estimate the beneficial rate using random effects generalized linear models having a binomial link and variance structure. Subjects will be included in the model as a random effect to account for within subject correlation. The model estimates and accompanying 95% confidence interval will be based on Wald statistics. The model will include all 180 observations from all 30 subjects (60 per treatment group) and will contain a fixed effect for treatment level. The potential difference in beneficial rates between treatments will be tested with the Wald test. If this model does not fit the data, we then will attempt to fit mixed Logistic regression models, in which case the parameter being estimated would be the odds of benefit and treatments compared using the odds ratio.

The previous analyses will be repeated for each of the possible changes between baseline and after 3 and 6 months of treatment and between baseline and the follow-up visit Longitudinal trend in benefit, and dependence on treatment level, will be examined using Markov State Transition models. False Discovery Rate will be considered when interpreting the results.

Other endpoints to be evaluated using descriptive statistical methods are:
Difference in C-peptide (Area Under the Curve [AUC]mean 0-120 min) during a Mixed Meal Tolerance Test (MMTT) between baseline and after 3 months of treatment, 6 months of treatment and the follow-up visit respectively for all treatments (Low dose daily oral GABA treatment, High dose daily oral GABA treatment, High dose oral GABA in combination with Alprazolam treatment).
Difference in C-peptide (Area Under the Curve [AUC]mean 0-120 min) during an MMTT between baseline and after 3 months of treatment, 6 months of treatment and the follow-up visit respectively for all treatments. Analyzed with an ultra-sensitive ELISA.
Difference in maximum stimulated C-peptide during an MMTT between baseline and after 3 months of treatment, 6 months of treatment and the follow-up visit respectively for all treatments.
Difference in maximum stimulated C-peptide during an MMTT between baseline and after 3 months of treatment, 6 months of treatment and the follow-up visit respectively for all treatments. Analyzed with an ultra-sensitive ELISA.
Difference between the treatments in difference in C-peptide (Area Under the Curve [AUC]mean 0-120 min) during an MMTT between baseline and after 3 months of treatment, 6 months of treatment and the follow-up visit.
Difference in glucagon (Area Under the Curve [AUC]mean 0-120 min) during an Hypoglycemic Clamp between baseline and 6 months of treatment for all treatments.
Difference between treatments in difference in glucagon (Area Under the Curve [AUC]mean 0-120 min) during an Hypoglycemic Clamp between baseline and 6 months of treatment.
Variables that indicate diabetes status such as plasma C-peptide, glucagon, proinsulin, proinsulin/C-peptide, glucagon like-peptide 1, lipids, Hemoglobin A1c (HbA1c) and insulin adjusted HbA1c (IDAAC). Daily exogenous insulin consumption, variability of blood sugar, and number of self-reported hypoglycemia.
Variables that indicate effects on the immune system such as serum autoantibodies (and isotypes) to GAD65 and Islet Antigen-2 and a general profiling of the immune cells in the blood.
Analysis of GABA plasma levels after 3 and 6 months of treatment and at the follow-up visit.
Measurements of patient QoL by questionnaire.

Formal statistical tests will be conducted based on availability of data. AUC values will be log transformed and differences estimated using normal-theory methods (e.g. t-test or ANOVA). Similarly, diabetes status and immune variables will be summarized with tables of mean, median, quartiles and confidence intervals. Tests of hypothesis will be conducted with either parametric (ANOVA) or nonparametric (Wilcoxon) methods. Categorical data (such as QoL and safety) will summarized with frequency tables and, when possible, treatments compared using Fisher's exact test or exact Chi-Square. All statistical tests will be conducted at the p<0.05 significance level.

Data Sets to be Analyzed
The Full Analysis Set (FAS) will consist of all randomized patients who have received at least one dose of study medication and have at least one post-baseline assessment and corresponding baseline measurement of any efficacy variable.
The Completers Set will consist of all patients in the FAS who have completed the corresponding study period and who have secondary efficacy data available.

The Per Protocol Set (PPS) will consist of all patients in the FAS who received GABA treatment according to the protocol and do not have any other major protocol violations which will affect the assessment of efficacy.
The Safety Set will consist of all randomized patients who received at least one dose of GABA.

The FAS is considered as the primary analysis dataset and will be used for all primary and secondary efficacy variables. The primary efficacy analyses will be repeated using the PPS and the completers set and these analyses will be regarded as sensitivity analyses. Any discrepancy between the results from the FAS and the PPS or the completers set will be analyzed and discussed.

The secondary variables will be analyzed based on FAS only.

Baseline presentations will be based on the safety set and on the FAS. Safety presentations will be based on the safety set.

Hypotheses and Statistical Methods

Analysis of Primary Endpoint Variable

Adverse Events:

AEs will be coded according to the Medical Dictionary for Regulatory Activities (MedDRA) system and tabulated by System Organ Class (SOC) and by preferred term.

A summary table will be presented with total number and No. of patients with:
AEs
serious AEs
related AEs and serious AEs
AE leading to discontinuation

AE will only be counted once within each patient on a preferred term level.

The total number of patients with at least one AE and the number of AEs will be derived and summarized by SOC and preferred term.

AEs will also be tabulated versus intensity and relationship to treatment (classified as related for outcomes “Unlikely related”, “Possible related” and “Probably related” and not related for outcome “Not related”. If a patient has more than one event classified with the same preferred term, then the worst intensity and the worst relationship will be used. In this table, patients having AEs will be identified by their patient number.

A treatment-emergent AE (TEAE) is an event emerging after treatment start or worsening during treatment. Hence, events with an onset time on or after the time of treatment start are treatment-emergent. Only TEAEs will be included in summary tables. Pre-treatment SAEs will be listed.

Treatment-emergent serious adverse events (TESAEs) will be listed separately.

In listings of AEs the relative day of occurrence will be given. Relative day will be calculated as (Date of first administration of IMP—AE start date)+1 for AEs occurring on date of first administration of IMP or later and as (Date of first administration of IMP—AE start date), otherwise.

Other Safety Endpoints:

All other safety endpoints will be presented using summary statistics. For laboratory measurements and vital signs change from baseline will also be summarized. Shift tables will present shifts for all endpoints for which normal/abnormal (NCS/CS) judgements exist.

Level of Significance, Multiple Comparisons and Multiplicity

The level of significance in the primary analyses will be 5%. No adjustment for multiple analyses will be made and p-values from analyses other than the primary analysis will be regarded as descriptive.

Adjustment for Covariates

Change from baseline analyses will be adjusted for baseline and all other analyses will be adjusted for randomization strata (C-peptide level).

Deviation(s) from the Statistical Analysis Plan

Any deviation from the final SAP should be stated in the final clinical report.

Results of Hypoglycemic Clamp Studies in Dose Escalation Part in Study Protocol 1

Only patients with c-peptide levels that were undetectable (in a standard assay, i.e. <0.01 nmol/L) are included (5 of 6) in the data below.

Plasma levels of glucagon, cortisol, epinephrine, growth hormone and glucose were measured at baseline (increased glucose in T1D subjects). Plasma glucose was first lowered to normoglycemic levels (5.5 mmol/L) and hormone levels monitored, and then to hypoglycemic levels (2.5 mmol/L) and hormone levels monitored. Difference was calculated as a measurement of the response to hypoglycemia.

The following effects of GABA treatment were found.
Baseline (no clamp) Cortisol and norepinephrine levels decrease with GABA treatment.
Normoglycemic (clamped to 5.5 mmol/L) levels of Cortisol, Glucagon, Growth hormone and norepinephrine decrease with GABA treatment.
Counter regulatory response (change in levels when clamping to 2.5 mmol/L from 5.5 mmol/L) of Cortisol, Epinephrine, Glucagon, and Growth hormone increased with GABA treatment.
Time in normal blood glucose range increased with GABA treatment.
Time below normal blood glucose range decreased with GABA treatment.
Time above normal blood glucose range decreased with GABA treatment.

Full details of the measured hormone levels are provided in the tables 10-14 below. In the tables below, visit 3 was performed in the pre-dosing period, visit 5 in the treatment period with 200 mg GABA per day, visit 7 in the treatment period with 600 mg GABA per day, and visit 9 in the treatment period with 1200 mg GABA per day. Plasma glucose levels are provided in Table 15

Blood glucose levels for all six patients in the dose escalation part of the study was measured during certain time intervals to determine time spent in, above, and below normal blood glucose range (4-10 mmol/L). The results are provided in Table 16 below. These results indicate that the counterregulatory response has been improved.

TABLE 10
Glucagon levels (pmol/L)

| C-peptide | Scheduled | | Observed value | | | | Change from baseline | | | | |
|---|---|---|---|---|---|---|---|---|---|---|---|
| at baseline | time point | Event | n | Mean (SD) | Median (Min, Max) | | n | Mean (SD) | | Median (Min, Max) | |
| Undetectable | Visit 3 | Baseline | 5 | 3.46 (0.62) | 3.80 | (2.6, 4.0) | — | — | | — | |
| | | 5.5 mmol/L | 5 | 2.19 (1.10) | 2.20 | (0.8, 3.8) | — | — | | — | |
| | | 2.5 mmol/L | 5 | 2.76 (0.40) | 2.80 | (2.3, 3.2) | — | — | | — | |
| | | Diff 2.5-5.5 mmol/L | 5 | 0.57 (1.29) | 0.70 | (−1.5, 2.1) | — | — | | — | |
| | Visit 5 | Baseline | 5 | 3.92 (1.03) | 3.60 | (2.8, 5.1) | 5 | 0.46 | (0.82) | 0.60 | (−0.8, 1.3) |
| | | 5.5 mmol/L | 5 | 2.08 (1.33) | 2.20 | (0.8, 3.7) | 5 | −0.11 | (0.92) | 0.00 | (−1.1, 1.3) |
| | | 2.5 mmol/L | 5 | 4.88 (2.44) | 4.80 | (2.7, 8.9) | 5 | 2.12 | (2.15) | 1.60 | (0.4, 5.8) |
| | | Diff 2.5-5.5 mmol/L | 5 | 2.80 (2.07) | 2.60 | (−0.3, 5.2) | 5 | 2.23 | (1.30) | 1.85 | (1.2, 4.5) |
| | Visit 7 | Baseline | 5 | 3.04 (0.66) | 3.10 | (2.0, 3.7) | 5 | −0.42 | (1.23) | −0.70 | (−2.0, 1.1) |
| | | 5.5 mmol/L | 5 | 1.53 (1.32) | 0.75 | (0.8, 3.8) | 5 | −0.66 | (0.71) | −0.60 | (−1.7, 0.0) |
| | | 2.5 mmol/L | 5 | 4.40 (1.26) | 4.20 | (3.2, 6.5) | 5 | 1.64 | (1.24) | 1.40 | (0.0, 3.4) |
| | | Diff 2.5-5.5 mmol/L | 5 | 2.87 (1.96) | 2.95 | (0.6, 5.8) | 5 | 2.30 | (1.68) | 2.10 | (0.6, 5.1) |
| | Visit 9 | Baseline | 5 | 4.42 (1.86) | 3.90 | (3.0, 7.6) | 5 | 0.96 | (1.71) | 0.50 | (−0.8, 3.8) |
| | | 5.5 mmol/L | 5 | 1.61 (1.18) | 0.75 | (0.8, 3.0) | 5 | −0.58 | (1.09) | −0.80 | (−1.7, 1.0) |
| | | 2.5 mmol/L | 5 | 5.92 (3.70) | 5.60 | (2.7, 12.0) | 5 | 3.16 | (3.58) | 3.30 | (0.0, 8.9) |
| | | Diff 2.5-5.5 mmol/L | 5 | 4.31 (4.33) | 2.60 | (−0.1, 11.3) | 5 | 3.74 | (4.23) | 3.30 | (−0.7, 10.6) |

TABLE 11
epinephrine levels (nmol/L)

| C-peptide | Scheduled | | Observed value | | | Change from baseline | | |
|---|---|---|---|---|---|---|---|---|
| at baseline | time point | Event | n | Mean (SD) | Median (Min, Max) | n | Mean (SD) | Median (Min, Max) |
| Undetectable | Visit 3 | Baseline | 5 | 0.150 (0.000) | 0.150 (0.15, 0.15) | — | — | — |
| | | 5.5 mmol/L | 5 | 0.150 (0.000) | 0.150 (0.15, 0.15) | — | — | — |
| | | 2.5 mmol/L | 5 | 0.840 (0.798) | 0.900 (0.15, 2.10) | — | — | — |
| | | Diff 2.5-5.5 mmol/L | 5 | 0.690 (0.798) | 0.750 (0.00, 1.95) | — | — | — |
| | Visit 5 | Baseline | 5 | 0.150 (0.000) | 0.150 (0.15, 0.15) | 5 | 0.000 (0.000) | 0.000 (0.00, 0.00) |
| | | 5.5 mmol/L | 5 | 0.150 (0.000) | 0.150 (0.15, 0.15) | 5 | 0.000 (0.000) | 0.000 (0.00, 0.00) |
| | | 2.5 mmol/L | 5 | 1.640 (1.014) | 1.200 (0.50, 2.90) | 5 | 0.800 (0.720) | 0.400 (0.30, 2.00) |
| | | Diff 2.5-5.5 mmol/L | 5 | 1.490 (1.014) | 1.050 (0.35, 2.75) | 5 | 0.800 (0.720) | 0.400 (0.30, 2.00) |
| | Visit 7 | Baseline | 5 | 0.150 (0.000) | 0.150 (0.15, 0.15) | 5 | 0.000 (0.000) | 0.000 (0.00, 0.00) |
| | | 5.5 mmol/L | 5 | 0.200 (0.112) | 0.150 (0.15, 0.40) | 5 | 0.050 (0.112) | 0.000 (0.00, 0.25) |
| | | 2.5 mmol/L | 5 | 1.400 (0.579) | 1.700 (0.60, 2.00) | 5 | 0.560 (0.585) | 0.800 (−0.40, 1.10) |
| | | Diff 2.5-5.5 mmol/L | 5 | 1.200 (0.630) | 1.550 (0.45, 1.85) | 5 | 0.510 (0.564) | 0.600 (−0.40, 1.10) |
| | Visit 9 | Baseline | 5 | 0.150 (0.000) | 0.150 (0.15, 0.15) | 5 | 0.000 (0.000) | 0.000 (0.00, 0.00) |
| | | 5.5 mmol/L | 5 | 0.150 (0.000) | 0.150 (0.15, 0.15) | 5 | 0.000 (0.000) | 0.000 (0.00, 0.00) |
| | | 2.5 mmol/L | 5 | 1.510 (1.070) | 1.200 (0.15, 2.60) | 5 | 0.670 (0.709) | 0.500 (0.00, 1.70) |
| | | Diff 2.5-5.5 mmol/L | 5 | 1.360 (1.070) | 1.050 (0.00, 2.45) | 5 | 0.670 (0.709) | 0.500 (0.00, 1.70) |

TABLE 12
Cortisol levels (nmol/L)

| C-peptide | Scheduled | | Observed value | | | | | Change from baseline | | | | |
|---|---|---|---|---|---|---|---|---|---|---|---|---|
| at baseline | time point | Event | n | Mean (SD) | | Median (Min, Max) | | n | Mean (SD) | | Median (Min, Max) | |
| Undetectable | Visit 3 | Baseline | 5 | 413.8 | (71.5) | 409.0 | (349, 522) | — | — | | — | |
| | | 5.5 mmol/L | 5 | 340.6 | (96.8) | 351.0 | (210, 444) | — | — | | — | |
| | | 2.5 mmol/L | 5 | 306.6 | (55.3) | 292.0 | (246, 392) | — | — | | — | |
| | | Diff 2.5-5.5 mmol/L | 5 | −34.0 | (134.4) | −105.0 | (−139, 182) | — | — | | — | |
| | Visit 5 | Baseline | 5 | 215.0 | (12.9) | 213.0 | (202, 236) | 5 | −198.8 | (80.0) | −196.0 | (−320, −115) |
| | | 5.5 mmol/L | 5 | 198.8 | (49.7) | 176.0 | (152, 277) | 5 | −141.8 | (117.2) | −179.0 | (−266, −3) |
| | | 2.5 mmol/L | 5 | 356.2 | (111.5) | 334.0 | (222, 502) | 5 | 49.6 | (119.6) | 40.0 | (−102, 223) |
| | | Diff 2.5-5.5 mmol/L | 5 | 157.4 | (150.5) | 162.0 | (5, 350) | 5 | 191.4 | (192.6) | 125.0 | (2, 489) |
| | Visit 7 | Baseline | 5 | 191.2 | (36.5) | 189.0 | (157, 251) | 5 | −222.6 | (87.4) | −217.0 | (−333, −100) |
| | | 5.5 mmol/L | 5 | 183.6 | (71.7) | 166.0 | (87, 275) | 5 | −157.0 | (98.0) | −185.0 | (−257, −5) |
| | | 2.5 mmol/L | 5 | 358.4 | (85.6) | 360.0 | (250, 487) | 5 | 51.8 | (112.1) | 36.0 | (−74, 208) |
| | | Diff 2.5-5.5 mmol/L | 5 | 174.8 | (134.9) | 194.0 | (21, 326) | 5 | 208.8 | (172.3) | 141.0 | (41, 465) |
| | Visit 9 | Baseline | 5 | 217.8 | (23.2) | 226.0 | (178, 235) | 5 | −196.0 | (60.4) | −191.0 | (−290, −125) |
| | | 5.5 mmol/L | 5 | 223.4 | (109.6) | 193.0 | (95, 363) | 5 | −117.2 | (144.4) | −115.0 | (−259, 83) |
| | | 2.5 mmol/L | 5 | 313.2 | (163.1) | 285.0 | (183, 585) | 5 | 6.6 | (197.3) | 29.0 | (−200, 306) |
| | | Diff 2.5-5.5 mmol/L | 5 | 89.8 | (195.5) | −10.0 | (−42, 426) | 5 | 123.8 | (260.6) | 83.0 | (−85, 565) |

TABLE 13
Growth hormone levels (μg/L)

| C-peptide | Scheduled | | Observed value | | | | | Change from baseline | | | | |
|---|---|---|---|---|---|---|---|---|---|---|---|---|
| at baseline | time point | Event | n | Mean (SD) | | Median (Min, Max) | | n | Mean (SD) | | Median (Min, Max) | |
| Undetectable | Visit 3 | Baseline | 5 | 0.155 | (0.165) | 0.100 | (0.03, 0.44) | — | — | | — | |
| | | 5.5 mmol/L | 5 | 2.912 | (3.554) | 0.520 | (0.06, 6.90) | — | — | | — | |
| | | 2.5 mmol/L | 5 | 7.072 | (12.861) | 1.330 | (0.10, 30.00) | — | — | | — | |
| | | Diff 2.5-5.5 mmol/L | 5 | 4.160 | (14.665) | −0.420 | (−5.97, 29.94) | — | — | | — | |
| | Visit 5 | Baseline | 5 | 0.630 | (0.926) | 0.080 | (0.06, 2.20) | 5 | 0.475 | (0.765) | 0.035 | (−0.02, 1.76) |
| | | 5.5 mmol/L | 5 | 2.110 | (4.094) | 0.070 | (0.06, 9.40) | 5 | −0.802 | (3.580) | 0.000 | (−6.83, 2.70) |
| | | 2.5 mmol/L | 5 | 11.632 | (13.994) | 4.000 | (0.06, 34.00) | 5 | 4.560 | (5.395) | 3.400 | (−0.87, 13.60) |
| | | Diff 2.5-5.5 mmol/L | 5 | 9.522 | (13.888) | 3.440 | (−0.01,33.94) | 5 | 5.362 | (3.385) | 4.000 | (2.09, 10.90) |
| | Visit 7 | Baseline | 5 | 2.253 | (4.779) | 0.140 | (0.03, 10.80) | 5 | 2.098 | (4.619) | 0.090 | (−0.08, 10.36) |
| | | 5.5 mmol/L | 5 | 1.100 | (2.181) | 0.160 | (0.06, 5.00) | 5 | −1.812 | (2.891) | −0.340 | (−6.84, 0.04) |
| | | 2.5 mmol/L | 5 | 17.140 | (12.989) | 11.700 | (5.80, 39.00) | 5 | 10.068 | (4.387) | 9.000 | (4.87, 16.87) |
| | | Diff 2.5-5.5 mmol/L | 5 | 16.040 | (13.667) | 10.820 | (5.74, 38.90) | 5 | 11.880 | (3.094) | 11.240 | (8.96, 17.09) |
| | Visit 9 | Baseline | 5 | 0.461 | (0.735) | 0.170 | (0.03, 1.77) | 5 | 0.306 | (0.754) | 0.100 | (−0.24, 1.63) |
| | | 5.5 mmol/L | 5 | 1.444 | (2.296) | 0.170 | (0.06, 5.40) | 5 | −1.468 | (3.112) | −0.210 | (−6.84, 1.00) |
| | | 2.5 mmol/L | 5 | 14.460 | (10.870) | 14.000 | (0.30, 27.00) | 5 | 7.388 | (9.868) | 7.900 | (−3.00, 21.67) |
| | | Diff 2.5-5.5 mmol/L | 5 | 13.016 | (11.351) | 8.600 | (0.24, 26.93) | 5 | 8.856 | (9.126) | 6.900 | (−3.01,21.88) |

TABLE 14
Norepinephrine (nmol(L)

| C-peptide | Scheduled | | Observed value | | | Change from baseline | | |
|---|---|---|---|---|---|---|---|---|
| at baseline | time point | Event | n | Mean (SD) | Median (Min, Max) | n | Mean (SD) | Median (Min, Max) |
| Undetectable | Visit 3 | Baseline | 5 | 1.80 (0.84) | 2.00 (0.7, 2.9) | — | — | — |
| | | 5.5 mmol/L | 5 | 1.94 (0.64) | 2.10 (1.0, 2.5) | — | — | — |
| | | 2.5 mmol/L | 5 | 2.26 (0.82) | 2.20 (1.2, 3.4) | — | — | — |
| | | Diff 2.5-5.5 mmol/L | 5 | 0.32 (0.33) | 0.20 (0.1, 0.9) | — | — | — |
| | Visit 5 | Baseline | 5 | 1.46 (0.35) | 1.50 (1.1, 2.0) | 5 | −0.34 (0.53) | −0.50 (−0.9, 0.5) |
| | | 5.5 mmol/L | 5 | 1.78 (0.52) | 1.60 (1.2, 2.5) | 5 | −0.16 (0.52) | 0.00 (−0.9, 0.5) |
| | | 2.5 mmol/L | 5 | 1.82 (0.41) | 1.90 (1.4, 2.3) | 5 | −0.44 (0.61) | −0.30 (−1.3, 0.2) |
| | | Diff 2.5-5.5 mmol/L | 5 | 0.04 (0.29) | 0.20 (−0.4, 0.3) | 5 | −0.28 (0.59) | 0.00 (−1.3, 0.1) |
| | Visit 7 | Baseline | 5 | 1.48 (0.70) | 1.20 (1.0, 2.7) | 5 | −0.32 (0.49) | −0.30 (−0.9, 0.4) |
| | | 5.5 mmol/L | 5 | 1.84 (0.34) | 2.00 (1.3, 2.1) | 5 | −0.10 (0.31) | −0.10 (−0.4, 0.3) |
| | | 2.5 mmol/L | 5 | 1.70 (0.27) | 1.70 (1.4, 2.1) | 5 | −0.56 (0.73) | −0.50 (−1.6, 0.3) |
| | | Diff 2.5-5.5 mmol/L | 5 | −0.14 (0.27) | −0.30 (−0.4, 0.2) | 5 | −0.46 (0.50) | −0.50 (−1.2, 0.0) |
| | Visit 9 | Baseline | 5 | 1.22 (0.35) | 1.00 (0.9, 1.6) | 5 | −0.58 (0.59) | −0.50 (−1.3, 0.2) |
| | | 5.5 mmol/L | 5 | 1.48 (0.50) | 1.40 (0.8, 2.2) | 5 | −0.46 (0.84) | −0.20 (−1.7, 0.4) |
| | | 2.5 mmol/L | 5 | 1.60 (0.42) | 1.60 (1.2, 2.2) | 5 | −0.66 (0.68) | −0.70 (−1.6, 0.0) |
| | | Diff 2.5-5.5 mmol/L | 5 | 0.12 (0.41) | 0.00 (−0.2, 0.8) | 5 | −0.20 (0.55) | −0.40 (−0.7, 0.7) |

TABLE 15
Glucose levels (mmol(L)

| C-peptide | Scheduled | | Observed value | | | | | Change from baseline | | | | |
|---|---|---|---|---|---|---|---|---|---|---|---|---|
| at baseline | time point | Event | n | Mean (SD) | | Median (Min, Max) | | n | Mean (SD) | | Median (Min, Max) | |
| Undetectable | Visit 3 | Baseline | 5 | 9.42 | (4.92) | 6.20 | (5.9, 16.8) | — | — | | — | |
| | | 5.5 mmol/L | 5 | 5.54 | (0.67) | 5.80 | (4.6, 6.1) | — | — | | — | |
| | | 2.5 mmol/L | 5 | 2.68 | (0.08) | 2.70 | (2.6, 2.8) | — | — | | — | |
| | | Diff 2.5-5.5 mmol/L | 5 | −2.86 | (0.68) | −3.20 | (−3.5, −1.9) | — | — | | — | |
| | Visit 5 | Baseline | 5 | 9.14 | (2.54) | 10.20 | (5.5, 11.7) | 5 | −0.28 | (5.40) | −0.40 | (−6.6, 5.5) |
| | | 5.5 mmol/L | 5 | 5.18 | (0.48) | 5.30 | (4.4, 5.7) | 5 | −0.36 | (0.91) | −0.10 | (−1.7, 0.6) |
| | | 2.5 mmol/L | 5 | 2.52 | (0.23) | 2.60 | (2.2, 2.8) | 5 | −0.16 | (0.27) | −0.20 | (−0.5, 0.2) |
| | | Diff 2.5-5.5 mmol/L | 5 | −2.66 | (0.53) | −2.90 | (−3.1, −1.8) | 5 | 0.20 | (1.06) | 0.10 | (−1.1, 1.5) |
| | Visit 7 | Baseline | 5 | 9.82 | (3.05) | 9.70 | (5.9, 13.9) | 5 | 0.40 | (3.49) | 1.70 | (−5.4, 3.5) |
| | | 5.5 mmol/L | 5 | 5.76 | (0.32) | 5.80 | (5.3, 6.1) | 5 | 0.22 | (0.69) | 0.00 | (−0.3, 1.4) |
| | | 2.5 mmol/L | 5 | 2.68 | (0.08) | 2.70 | (2.6, 2.8) | 5 | 0.00 | (0.10) | 0.00 | (−0.1, 0.1) |
| | | Diff 2.5-5.5 mmol/L | 5 | −3.08 | (0.28) | −3.20 | (−3.4, −2.7) | 5 | −0.22 | (0.66) | −0.10 | (−1.3, 0.3) |
| | Visit 9 | Baseline | 5 | 9.42 | (3.64) | 7.90 | (5.4, 13.8) | 5 | 0.00 | (5.08) | 1.30 | (−6.8, 6.5) |
| | | 5.5 mmol/L | 5 | 5.30 | (0.27) | 5.30 | (5.0, 5.7) | 5 | −0.24 | (0.76) | −0.40 | (−1.1, 0.6) |
| | | 2.5 mmol/L | 5 | 2.70 | (0.12) | 2.70 | (2.5, 2.8) | 5 | 0.02 | (0.18) | 0.00 | (−0.2, 0.2) |
| | | Diff 2.5-5.5 mmol/L | 5 | −2.60 | (0.28) | −2.60 | (−3.0, −2.2) | 5 | 0.26 | (0.87) | 0.60 | (−0.7, 1.3) |

TABLE 16
Time spent below, within, and above normal blood glucose range

| Time interval | Statistic | Time below range (%) | | Time within range (%) | | Time above range (%) | |
|---|---|---|---|---|---|---|---|
| Inclusion to day | n | 6 | | 6 | | 6 | |
| before baseline visit | Mean (SD) | 10.4 | (7.6) | 39.2 | (17.4) | 50.4 | (24.5) |
| | Median (Min, Max) | 8.9 | (2.3, 21.1) | 33.9 | (19.8, 63.0) | 57.3 | (15.9, 75.6) |
| Day 3 to 4 | n | 6 | | 6 | | 6 | |
| | Mean (SD) | 14.8 | (12.3) | 56.6 | (20.2) | 28.7 | (13.5) |
| | Median (Min, Max) | 14.3 | (0.0, 35.5) | 62.0 | (24.0, 76.0) | 28.8 | (5.0, 43.0) |
| Day 6 to 7 | n | 6 | | 6 | | 6 | |
| | Mean (SD) | 4.8 | (2.5) | 57.6 | (11.2) | 37.7 | (13.1) |
| | Median (Min, Max) | 4.0 | (2.5, 9.5) | 59.5 | (41.0, 67.5) | 36.5 | (23.0, 56.5) |
| Day 9 | n | 6 | | 6 | | 6 | |
| | Mean (SD) | 5.5 | (9.6) | 55.0 | (20.2) | 39.5 | (25.1) |
| | Median (Min, Max) | 2.5 | (0.0, 25.0) | 47.0 | (34.0, 90.0) | 50.0 | (8.0, 66.0) |

### REFERENCES

All references cited herein are expressly incorporated by reference in their entirety.
Antony et al. (2010). Decreased GABA receptor binding in the cerebral cortex of insulin induced hypoglycemic and streptozotocin induced diabetic rats. Neurochem Res., 35(10), 15-16-1521.
Chan et al. (May 2008). Diabetes, 57(5), 1363-1370.
Cryer, P. E. (2001). Hypoglycemia-associated autonomic failure in diabetes. Am J Physiol Endocrinol Metab., 281(6), E1115-E1121.
Davis et al. (1992). Further defects in counterregulatory responses induced by recurrent hypoglycemia in IDDM. Diabetes, 41(10), 1335-1340.
Giordano et al. (2003). Alprazolam (a benzodiazepine activating GABA receptor) reduces the neuroendocrine responses to insulin-induced hypoglycemia in humans. Clin Endocrinol (Oxf)., 59(3), 314-320.
Hedrington et al. (2010). Effects of Antecedent GABAA Activation With Alprazolam on Counterregulatory Responses to Hypoglycemia in Healthy Humans. Diabetes, 59(4), 1074-1081.
Heller, S., & Cryer, P. (1991). Reduced neuroendocrine and symptomatic responses to subsequent hypoglycemia after 1 episode of hypoglycemia in nondiabetic humans. Diabetes, 40(2), 223-226.
McCrimmon, R. J., & Sherwin, R. S. (2010). Hypoglycemia in Type 1 Diabetes. Diabetes, 59(10), 2333-2339.
Ryan, E. A. (2004). Diabetes, 53(4), 955-962.
White, J. R. (2007). The Contribution of Medications to Hypoglycemia Unawareness. Diabetes Spectrum, 20(2), 77-80.

1. A method for preventing, or reducing risk of, hypoglycemia in a subject, the method comprising administering gamma-amino butyric acid (GABA) or a GABA receptor agonist to the subject.

2. The method of claim 1, wherein the GABA, or GABA receptor agonist is administered to the subject in combination with at least one compound selected from the group consisting of Positive Allosteric Modulators of a GABA-receptor (PAM), Selective Serotonin Reuptake Inhibitors (SSRIs) and dehydroepiandrosterone.

3. The method of claim 1, wherein the subject suffers from type 1 diabetes, type 2 diabetes, insulin-dependent diabetes mellitus, and/or impaired endogenous insulin production.

4. The method of claim 1, wherein the subject has insulin-induced hypoglycemia.

5. The method of claim 1, wherein the subject has non-insulin-induced hypoglycemia in said subject.

6. The method of claim 5, wherein the non-insulin-induced hypoglycemia is induced by sulfonylurea, beta blockers, Selective Serotonin Re-uptake Inhibitors (SSRI:s), exercise, islet transplants, and/or bariatric surgery.

7. The method of claim 5, wherein the non-insulin-induced hypoglycemia is induced by sulfonylurea.

8-9. (canceled)

10. The method of claim 1, wherein the subject has a defect in hypoglycemia counterregulation.

11. The method of claim 10, wherein the Gamma-amino butyric acid (GAB A), or a GABA receptor agonist, is administered to the subject in combination with at least one compound selected from the group consisting of Positive Allosteric Modulators of a GABA-receptor (PAM), Selective Serotonin Reuptake Inhibitors (SSRIs) and dehydroepiandrosterone.

12. The method of claim 1, wherein the method results in an increase in the time spent in normal blood glucose range (4-10 mmol/L) or target blood glucose range (4-8 mmol/L) by the subject.

13. The method of claim 10, wherein said subject suffers type 1 diabetes, type 2 diabetes, insulin-dependent diabetes mellitus, and/or impaired endogenous insulin production.

14. The method of claim 1, wherein the GABA, or the GABA receptor agonist is administered to the subject one hour or less before a meal or going to bed.

15. A pharmaceutical composition comprising insulin and GABA, or a GABA receptor agonist, and optionally at least one compound selected from the group consisting of Positive Allosteric Modulators of a GABA-receptor (PAM), Selective Serotonin Reuptake Inhibitors (SSRIs) and dehydroepiandrosterone.

16. A pharmaceutical composition comprising sulfonylurea and GABA, or a GABA receptor agonist, and optionally at least one compound selected from the group consisting of Positive Allosteric Modulators of a GABA-receptor (PAM), Selective Serotonin Reuptake Inhibitors (SSRIs) and dehydroepiandrosterone.
